(19) United States
(12) Patent Application Publication
(10) Pub. No.: US 2020/0385739 A1
(43) Pub. Date: Dec. 10, 2020

(54) COMPOSITIONS AND METHODS FOR ENHANCING WEIGHT LOSS

(71) Applicant: The Children's Hospital of Philadelphia, Philadelphia, PA (US)

(72) Inventors: Hakon Hakonarson, Malvern, PA (US); Rahul Pandey, Wallingford, PA (US); Marina Bakay, Parkesburg, PA (US); Heather Hain, Ambler, PA (US)

(21) Appl. No.: 16/644,347

(22) PCT Filed: Sep. 7, 2018
(86) PCT No.: PCT/US18/50027

§ 371 Date: Mar. 4, 2020

Related U.S. Application Data
(60) Provisional application No. 62/555,631, filed on Sep. 7, 2017.

(51) Int. Cl. C12N 15/113; A61K 45/06; A61P 3/04

(52) U.S. Cl. CPC C12N 15/1138; A61P 3/04; A61K 45/06

(57) ABSTRACT

Compositions and methods for the detection and treatment of obesity and other neurological disorders are provided.

This invention claims priority to U.S. Provisional Application No. 62/555,631 filed Sep. 7, 2017, the entire contents being incorporated herein by reference as though set forth in full.

### FIELD OF THE INVENTION

This invention relates to the fields of genetics and metabolism. More specifically, the invention provides compositions and methods for enhancing weight loss in subjects in need thereof by modulating CLEC16A expression levels.

### BACKGROUND OF THE INVENTION

Several publications and patent documents are cited throughout the specification in order to describe the state of the art to which this invention pertains. Each of these citations is incorporated by reference herein as though set forth in full.

Clec16a has been identified as a disease susceptibility gene for type 1 diabetes, multiple sclerosis, and adrenal dysfunction, to name a few (14 autoimmune diseases total have been associated by us and others to this autoimmune gene). Clec16a is a membrane-associated endosomal protein that interacts with E3 ubiquitin ligase Nrdp1. Loss of Clec16a leads to an increase in the Nrdp1 target Parkin, a master regulator of mitophagy. Islets from mice with pancreas-specific deletion of Clec16a have abnormal mitochondria with reduced oxygen consumption and ATP concentration, both of which are required for normal p cell function. Indeed, pancreatic Clec16a is required for normal glucose-stimulated insulin release. Moreover, patients harboring a diabetogenic SNP in the Clec16a gene have reduced islet Clec16a expression and reduced insulin secretion. Thus, Clec16a controls β cell function and prevents diabetes by controlling mitophagy. This pathway could be targeted for prevention and control of diabetes and may extend to the pathogenesis of other Clec16a- and Parkin-associated diseases.

It is clear from the foregoing, that therapeutic agents which specifically target Clec16a should have efficacy for the treatment of a variety of disorders, including diabetes, obesity and certain neurological disorders.

### SUMMARY OF THE INVENTION

In accordance with the present invention, a method of management for obesity in a subject in need thereof comprising administering to said subject a therapeutic agent in amount effective to partially reduce CLEC16A expression, thereby managing or reducing obesity is disclosed. In certain embodiments, the therapeutic agent modulates signaling mediated via the CLEC16A gene product. In other embodiments, the therapeutic agent is selected from the group consisting of a small molecule, an antibody, a protein, an oligonucleotide, or an siRNA molecule. The therapeutic agent can also be one or more of an autophagy inhibitor, an inhibitor of the Jak-Stat pathway or an mTor inhibitor.

In some embodiments, the agent is delivered to a cell selected from an adipose cell or an insulin-producing beta cell. In other embodiments, the agent modulates natural killer cell activity.

In other embodiments, the therapeutic agent comprises at least one siRNA molecule provided in Table 1. The therapeutic agent can be targeted to adipose cells, natural killer cells for example. In certain embodiments, the therapeutic agent modulates signaling in an insulin-producing beta cell.

In another aspect, the invention provides a pharmaceutical composition comprising a therapeutic agent which partially inhibits CLEC16A expression in a target cell. In certain embodiments, an siRNA composition comprising at least one nucleotide sequence selected from the group listed in Table 1 in a pharmaceutically acceptable carrier for delivery to a patient. Thus, another aspect of the invention entails a method of partially inhibiting the expression of CLEC16A in a patient comprising administering to said patient at least one siRNA molecule that directs cleavage of a target CLEC16A mRNA sequence present in said patient. Such siRNAs can be used alone or in combination with other siRNAs or agents conventionally used for the management of obesity. Suitable agents, include, without limitation, autophagy inhibitors, mTor inhibitors and Jak-stat pathway inhibitors.

### BRIEF DESCRIPTION OF THE DRAWINGS

FIG. 1. Percent body weight from initial body weight over time. Mice were weighed 3 times per week during the study.

FIG. 2. Tamoxifen-inducible UBC-Cre-Clec16a^{loxP} male and female mice exhibit complete loss of body and visceral fat.

FIG. 3. Food Intake (per gram of body weight in UBC-Cre-Clec16a^{loxP} KO mice as compared to controls.

FIG. 4. Phosphorylation of HSL at Ser563, Ser659 and Ser660 in control and Clec16a KO mice adipose tissue.

FIG. 5. The mRNA expression of lipid catabolism genes (Cpt1b, Ppara), adipogenic genes (Pparg andAdipoq) and thermogenic genes (Ucp1 and Cidea) from gWAT of control and KO mice determined by qPCR (n=8).

FIG. 6. Lipid analysis (cholesterol, triglyceride and free fatty acid) from Clec16a KO vs. control mice serum.

FIG. 7a. Clec16a KO mice exhibit decreased Adiponectin, leptin and LDL-R in comparison to control. FIG. 7b. UBC-Cre-Clec16a^{loxP} KO mice exhibit increased cytokines, chemokines, growth factors and other soluble proteins in adipose tissue compared to control. FIG. 7c. UBC-Cre-Clec16a^{loxP} KO mice exhibit increased cytokines, chemokines, growth factors and other soluble proteins in adipose tissue compared to control. FIG. 7d. UBC-Cre-Clec16a^{loxP} KO mice exhibit increased cytokines/chemokines in plasma compared to control.

FIG. 8. High constitutive expression of IL-16 precursor in Clec16a KO splenocytes and release of bioactive IL-16 by active caspase-3.

FIG. 9. Decreased expression of SOCS1 and SOCS3 in Clec16a KO splenocytes.

FIG. 10. Tofacitinib, pan JAK/STAT inhibitor, partially rescues the lipodystrophic phenotype and improves survival of UBC-Cre-Clec16a^{loxP} KO mice.

FIG. 11. Predominant Th-1 Cytokine/chemokine in Clec16a KO and rescue with Tofacitinib. The representative graph is quantification of cytokines and chemokine from plasma of Control (Vehicle), KO and KO+Tofacitinib inhibitor treated mice using the Mouse Cytokine Array panel.

FIG. 12. ANA-9 line Immunoblot assay. Lane 1 positive control is showing all the antigens. Lane 2 &3 are probed with sera from control mice; lane 4-8 are probed with sera of Clec16a KO mice.

FIG. 13. Serum Immunoglobulin Isotyping. ELISA was performed to evaluate changes in serum Immunoglobulins isotypes, isotypes and IgG subclasses with control mice and Clec16a KO mice sera (n=10).

FIG. 14. Clec16a knockout induces disability in mice. KO mice exhibit abnormal neurons in the dorsal root ganglia due to dysregulated mitophagy. Activated microglia with inflammation in the spinal cord dorsal columns and loss of cerebellar Purkinje cells is evident.

### DETAILED DESCRIPTION OF THE INVENTION

In view of the prominent role of CLEC16A SNP associations in variety of autoimmune disorders, we generated a novel whole-body Clec16a inducible knockdown (KD) mouse, in which with tamoxifen treatment CLEC16A expression could be turned off in all organs at desired time points (UBC-Cre-Clec6a^{loxP}). In our study, we discovered that turning off Clec16a in 8-10 week-old mice leads to severe weight loss (20%) accompanied by a systemic inflammatory response. Complete KO resulted in additional autoimmune and neurologic phenotypes, including decreased numbers of Schwann cells, the cells which insulate (myelinate) individual nerve fibers (axons); abnormal axons; and myelin debris. In addition, we found pathological changes in Dorsal Root Ganglion (DRG) neurons with accumulated vacuoles and abnormal (swollen or dying) mitochondria—all in concordance with previously published discovery by us, that CLEC16A controls mitophagy and absence of this protein leads to accumulation of unhealthy mitochondria in pancreatic beta cells (Soleimanpour et al., 2014). However, partial knock down of the gene resulted in weight loss without evidence for additional phenotypes, which let us consider CLEC16A (or its pathway) as a target for weight reduction therapy.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The phrase “Type 1 diabetes (T1D)” refers to a chronic (lifelong) disease that occurs when the pancreas produces too little insulin to regulate blood sugar levels appropriately. T1D, often called juvenile or insulin-dependent diabetes results from altered metabolism of carbohydrates (including sugars such as glucose), proteins, and fats. In type 1 diabetes, the beta cells of the pancreas produce little or no insulin, the hormone that allows glucose to enter body cells. Once glucose enters a cell, it is used as fuel. Without adequate insulin, glucose builds up in the bloodstream instead of going into the cells. The body is unable to use this glucose for energy despite high levels in the bloodstream, leading to increased hunger. In addition, the high levels of glucose in the blood cause the patient to urinate more, which in turn causes excessive thirst. Within 5 to 10 years after diagnosis, the insulin-producing beta cells of the pancreas are completely destroyed, and no more insulin is produced.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting CLEC 16A mRNA may be between 15-35 nucleotides in length, and more typically about 21 nucleotides in length. A list of candidate siRNAs directed to CLEC 16A are provided in Table 1.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, peptide-tethering, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the T1D specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the T1D specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

Terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, small molecules, antibodies, peptides, peptide/DNA complexes, and any nucleic acid based molecule, for example an oligo, which exhibits the capacity to modulate the activity of the CLEC16A encoding nucleic acids described herein or the encoded protein. Agents can be evaluated for potential biological activity by inclusion in screening assays described herein below.

The term “modulate” as used herein refers increasing or decreasing. For example, the term modulate refers to the ability of a compound or test agent to interfere with signaling or activity of a gene or protein of the present invention. Therefore, modulating the signaling mediated by CLEC16A means that an agent or compound inhibits or enhances the activity of the protein encoded by the gene. This includes altering lipolysis activity, mitophagy, the activity of natural killer cells, and rates of autoimmune beta cell destruction.

### Pharmaceutical Agents and Therapies

The elucidation of the role played by CLEC16A described herein in cellular metabolism facilitates the development of pharmaceutical compositions useful for treatment and diagnosis of obesity and certain neurological disorders. For example, CLEC16A plays an important role in autophagy. Accordingly, autophagy inhibitors have utility in the present invention. These include, without limitation, SP600125, U0126, 3-Methyladenine, Bafilomycin A1, Chloroquine, LY294002, SB202190, SB203580, SC79 and wortmannin which may act to rescue loss of CLEC16A function in affected individuals.

Jak-Stat inhibitors can also be used to advantage to partially inhibit CLEC16A. Such inhibitors include without limitation,

Ruxolitinib (INCB018424) is the first potent, selective, JAK1/2 inhibitor to enter the clinic with IC50 of 3.3 nM/2.8 nM in cell-free assays, >130-fold selectivity for JAK1/2 versus JAK3. Science, 2018, 10(436) NAT MATER, 2017, 10.1038/NMAT5024 Nat Med, 2015, 10.1038/nm.4013

Tofacitinib citrate (CP-690550 citrate) is a novel inhibitor of JAK with IC50 of 1 nM, 20 nM and 112 nM against JAK3, JAK2, and JAK1, respectively. Cancer Discov, 2012, 2(7):591-7 Nat Cell Biol, 2015, 17(1):57-67 Blood, 2014, 124(5):761-70

AZD1480 is a novel ATP-competitive JAK2 inhibitor with IC50 of 0.26 nM in a cell-free assay, selectivity against JAK3 and Tyk2, and to a smaller extent against JAK1. Phase 1. Nat Cell Biol, 2015, 17(1):57-67 Blood, 2014, 123(10):1516-24 Leukemia, 2012, 26(4):708-15

Fedratinib (SAR302503, TG101348) is a selective inhibitor of JAK2 with IC50 of 3 nM in cell-free assays, 35- and 334-fold more selective for JAK2 versus JAK1 and JAK3. Phase 2. Cell, 2015, 162(2):441-51 Blood, 2014, 123(20):3175-84 J Thorac Oncol, 2016, 11(1):62-71

AT9283 is a potent JAK2/3 inhibitor with IC50 of 1.2 nM/1.1 nM in cell-free assays; also potent to Aurora A/B, Abl(T315I). Phase 2. Cell Stem Cell, 2012, 11(2):179-94 Cancer Res, 2013, 73(20):6310-22 Cancer Lett, 2013, 341(2):224-30

Ruxolitinib Phosphate is the phosphate salt form of ruxolitinib, an orally bioavailable Janus-associated kinase (JAK) inhibitor with potential antineoplastic and immunomodulating activities.

Itacitinib(INCB39110) is an orally bioavailable inhibitor of Janus-associated kinase 1 (JAK1) with potential antineoplastic activity.

PF-06651600 is a potent and irreversible JAK3-selective inhibitor with an IC50 of 33.1 nM but without activity (IC50>10 000 nM) against JAK1, JAK2, and TYK2.

FM-381 is a JAK3 specific reversible covalent inhibitor with IC50 of 127 pM for JAK3 and demonstrates 400-, 2,700- and 3,600-fold selectivity over JAK1, JAK2, and TYK2, respectively.

Momelotinib (CYT387) is an ATP-competitive inhibitor of JAK1/JAK2 with IC50 of 11 nM/18 nM, ˜10-fold selectivity versus JAK3. Phase 3. Nat Cell Biol, 2015, 17(1):57-67 Blood, 2012, 120(19):4093-103 J Clin Invest, 2014, 124(12):5263-74

Tofacitinib (CP-690550, Tasocitinib) is a novel inhibitor of JAK3 with IC50 of 1 nM in cell-free assays, 20- to 100-fold less potent against JAK2 and JAK1. Blood, 2014, 124(5):761-70 Blood, 2012, 120(4):709-19 Blood, 2011, 118(14):3911-21

WP1066 is a novel inhibitor of JAK2 and STAT3 with IC50 of 2.30 M and 2.43 M in HEL cells; shows activity to JAK2, STAT3, STAT5, and ERK1/2 not JAK1 and JAK3. Phase 1. Int J Cancer, 2014, 135(2):282-94 Exp Neurol, 2015, 271:445-56 J Biol Chem, 2013, 288(36):26167-76

TG101209 is a selective JAK2 inhibitor with IC50 of 6 nM, less potent to Flt3 and RET with IC50 of 25 nM and 17 nM in cell-free assays, ˜30-fold selective for JAK2 than JAK3, sensitive to JAK2V617F and MPLW515L/K mutations. Leukemia, 2014, 28(7):1519-28 Cancer Lett, 2013, 341(2):224-30 ACS Chem Biol, 2014, 9(5):1160-71

Gandotinib (LY2784544) is a potent JAK2 inhibitor with IC50 of 3 nM, effective in JAK2V617F, 8- and 20-fold selective versus JAK1 and JAK3. Phase 2. Cancer Lett, 2013, 341(2):224-30 Gastric Cancer, 2016, 19(1):53-62 Eur J Pharmacol, 2015, 765:188-97

NVP-BSK805 2HCl is a potent and selective ATP-competitive JAK2 inhibitor with IC50 of 0.5 nM, >20-fold selectivity towards JAK1, JAK3 and TYK2. Cancer Lett, 2013, 341(2):224-30 PLoS One, 2013, 8(5):e63301

Baricitinib (LY3009104, INCB028050) is a selective JAK1 and JAK2 inhibitor with IC50 of 5.9 nM and 5.7 nM in cell-free assays, ˜70 and ˜10-fold selective versus JAK3 and Tyk2, no inhibition to c-Met and Chk2. Phase 3. Nat Cell Biol, 2014, 17(1):57-67 Br J Haematol, 2017, 177(2):271-282

AZ 960 is a novel ATP competitive JAK2 inhibitor with IC50 and Ki of <3 nM and 0.45 nM, 3-fold selectivity of AZ960 for JAK2 over JAK3. Cancer Lett, 2013, 341(2):224-30

CEP33779 is a selective JAK2 inhibitor with IC50 of 1.8 nM, >40- and >800-fold versus JAK1 and TYK2. Biochem Pharmacol, 2014, 91(2):144-56 Biosci Rep, 2017, 37(4)

Pacritinib (SB1518) is a potent and selective inhibitor of Janus Kinase 2 (JAK2) and Fms-Like Tyrosine Kinase-3 (FLT3) with IC50s of 23 and 22 nM in cell-free assays, respectively. Phase 3.

WHI-P154 is a potent JAK3 inhibitor with IC50 of 1.8 μM, no activity against JAK1 or JAK2, also inhibits EGFR, Src, Abl, VEGFR and MAPK, prevents Stat3, but not Stat5 phosphorylation. Oncol Rep, 2017, 37(1):66-76

XL019 is a potent and selective JAK2 inhibitor with IC50 of 2.2 nM, exhibiting >50-fold selectivity over JAK1, JAK3 and TYK2. Phase 1.

S-Ruxolitinib is the chirality of INCB018424, which is the first potent, selective, JAK1/2 inhibitor to enter the clinic with IC50 of 3.3 nM/2.8 nM, >130-fold selectivity for JAK1/2 versus JAK3. Phase 3. Clin Cancer Res, 2015, 21(16):3740-9 Blood Cancer J, 2017, 7(6):e572 Sci Rep, 2016, 6:28473

ZM 39923 HCl is an JAK1/3 inhibitor with pIC50 of 4.4/7.1, almost no activity to JAK2 and modestly potent to EGFR; also found to be sensitive to transglutaminase.

Peficitinib (ASP015K, JNJ-54781532) is an orally bioavailable JAK inhibitor. Phase 3.

Filgotinib (GLPG0634) is a selective JAK1 inhibitor with IC50 of 10 nM, 28 nM, 810 nM, and 116 nM for JAK1, JAK2, JAK3, and TYK2, respectively. Phase 2.

Decernotinib (VX-509) is a potent and selective JAK3 inhibitor with Ki of 2.5 nM, >4-fold selectivity over JAK1, JAK2, and TYK2, respectively. Phase 2/3.

BMS-911543 is a potent and selective inhibitor of JAK2 with IC50 of 1.1 nM, ˜350-, 75- and 65-fold selective to JAK1, JAK3 and TYK2, respectively. Phase 1/2.

FLLL32 is a potent JAK2/STAT3 inhibitor with IC50 of <5 M. Cancer Sci, 2016, 107(7):944-54 Eur Rev Med Pharmacol Sci, 2017, 21(13):3005-3011

Curcumol is a pure monomer isolated from Rhizoma Curcumaeis with antitumor activities.

GLPG0634 analogue is a selective JAK1 inhibitor with IC50 of 10 nM, 28 nM, 810 nM, and 116 nM for JAK1, JAK2, JAK3, and TYK2, respectively. Phase 2.

Oclacitinib (PF 03394197) is a novel inhibitor of JAK family members with IC50 ranging from 10 to 99 nM and JAK1-dependent cytokines with IC50 ranging from 36 to 249 nM. It does not inhibit a panel of 38 non-JAK kinases.

Cerdulatinib (PRT-062070) is an oral active, multi-targeted tyrosine kinase inhibitor with IC50 of 12 nM/6 nM/8 nM/0.5 nM and 32 nM for JAK1/JAK2/JAK3/TYK2 and Syk, respectively. Also inhibits 19 other tested kinases with IC50 less than 200 nM. J Immunol, 2016, 197(7):2948-57

Go6976 is a potent PKC inhibitor with IC50 of 7.9 nM, 2.3 nM, and 6.2 nM for PKC (Rat brain), PKCa, and PKCP 1, respectively. Also a potent inhibitor of JAK2 and Flt3. Cell Signal, 2016, 28(9):1422-31 Infect Immun, 2017, e00087-17.

mTor inhibitors also have utility in the methods disclosed herein and include, for example,

Dactolisib (BEZ235, NVP-BEZ235) is a dual ATP-competitive PI3K and mTOR inhibitor for p110α/γ/δ/β and mTOR(p70S6K) with IC50 of 4 nM/5 nM/7 nM/75 nM/6 nM in cell-free assays, respectively. Inhibits ATR with IC50 of 21 nM in 3T3TopBP1-ER cell. Nature, 2017, 728-732 Nature, 2012, 487(7408):505-9 Nat Med, 2015, 10.1038/nm.3855

Rapamycin (Sirolimus) is a specific mTOR inhibitor with IC50 of ˜0.1 nM HEK293 cells. Nature, 2016, 539(7629):437-442 Nat Genet, 2014, 46(4):364-70 Cancer Cell, 2011, 19(6):792-804

Everolimus (RAD001) is an mTOR inhibitor of FKBP12 with IC50 of 1.6-2.4 nM in a cell-free assay. Nat Med, 2015, 10.1038/nm.3855 Cell, 2016, 164(1-2):293-309 Cell, 2016, 164(1-2):293-309

AZD8055 is a novel ATP-competitive mTOR inhibitor with IC50 of 0.8 nM in MDA-MB-468 cells with excellent selectivity (˜1,000-fold) against PI3K isoforms and ATM/DNA-PK. Phase 1. Nat Med, 2015, 10.1038/nm.3855 Cancer Cell, 2015, 27(1):97-108 Cancer Cell, 2015, 27(4):533-46

Temsirolimus (CCI-779, NSC 683864) is a specific mTOR inhibitor with IC50 of 1.76 M in a cell-free assay. Autophagy, 2011, 7(2):176-87 Cancer Res, 2014, 74(14):3947-58 Mol Cancer, 2014, 13(1):159

PI-103

PI-103 is a multi-targeted PI3K inhibitor for p110α/β/δ/γ with IC50 of 2 nM/3 nM/3 nM/15 nM in cell-free assays, less potent to mTOR/DNA-PK with IC50 of 30 nM/23 nM. Cell, 2013, 153(4):840-54 Leukemia, 2013, 27(3):650-60 Leukemia, 2012, 26(5):927-33

KU-0063794 is a potent and highly specific dual-mTOR inhibitor of mTORC1 and mTORC2 with IC50 of ˜10 nM in cell-free assays; no effect on PI3Ks. Cell Stem Cell, 2012, 10(2):210-7 Circ Res, 2010, 107(10):1265-74 Oncogene, 2013, 10.1038/onc.2013.509

Torkinib (PP242) is a selective mTOR inhibitor with IC50 of 8 nM in cell-free assays; targets both mTOR complexes with >10- and 100-fold selectivity for mTOR than PI3Kδ or PI3Kα/β/γ, respectively. Science, 2016, 353(6302):929-32 Nat Chem Biol, 2013, 9(11):708-14 J Clin Invest, 2015, 10.1172/JCI78018

Tacrolimus (FK506) is a 23-membered macrolide lactone, it reduces peptidyl-prolyl isomerase activity in T cells by binding to the immunophilin FKBP12 (FK506 binding protein) creating a new complex. Biochim Biophys Acta, 2015, 1853(10 Pt A):2684-96 Biochim Biophys Acta, 2012, 1833(3):652-62 Biomed Pharmacother, 2013, 67(6):469-73

Ridaforolimus (Deforolimus, MK-8669) is a selective mTOR inhibitor with IC50 of 0.2 nM in HT-1080 cell line; while not classified as a prodrug, mTOR inhibition and FKBP12 binding is similar to rapamycin. Phase 3. Gynecol Oncol, 2016, 141(3):570-9 J Lipid Res, 2014, 55(5):919-28 Mol Pharmaco, 2013, 84(1):104-13

Sapanisertib (INK 128, MLN0128) is a potent and selective mTOR inhibitor with IC50 of 1 nM in cell-free assays; >200-fold less potent to class I PI3K isoforms, superior in blocking mTORC1/2 and sensitive to pro-invasion genes (vs Rapamycin). Phase 1. Cancer Discov, 2014, 4(5):554-63 Cell Rep, 2015, 11(3):446-59 Cell Communication and Signaling, 2015, 13:15

Voxtalisib (SAR245409, XL765) Analogue is a dual inhibitor of mTOR/PI3K, mostly for p110γ with IC50 of 9 nM; also inhibits DNA-PK and mTOR. Phase 1/2. Cell Rep, 2015, 11(3):446-59 Mol Cancer Res, 2014, 12(5):703-13 Endocrinology, 2013, 154(3):1247-59

Torin 1 is a potent inhibitor of mTORC1/2 with IC50 of 2 nM/10 nM in cell-free assays; exhibits 1000-fold selectivity for mTOR than PI3K. Cancer Discov, 2016, 6(7):727-39 Elife, 2015, 4 Am J Pathol, 2014, 184(1):214-29

Omipalisib (GSK2126458, GSK458) is a highly selective and potent inhibitor of p110α/β/δ/γ, mTORC1/2 with Ki of 0.019 nM/0.13 nM/0.024 nM/0.06 nM and 0.18 nM/0.3 nM in cell-free assays, respectively. Phase 1. Proc Natl Acad Sci USA, 2013, 110(10):4015-20 Neuro Oncol, 2016, 18(4):528-37 Mol Cancer Ther, 2015, 14(2):429-39

OSI-027 is a selective and potent dual inhibitor of mTORC1 and mTORC2 with IC50 of 22 nM and 65 nM in cell-free assays, and more than 100-fold selectivity observed for mTOR than PI3Kα, PI3Kβ, PI3Kγ or DNA-PK. Phase 1. Cell Rep, 2015, 11(3):446-59 Eur J Cancer, 2013, 74:41-9 Br J Cancer, 2016, 114(6):650-8

PF-04691502 is an ATP-competitive PI3K(α/β/δ/γ)/mTOR dual inhibitor with Ki of 1.8 nM/2.1 nM/1.6 nM/1.9 nM and 16 nM in cell-free assays, little activity against either Vps34, AKT, PDK1, p70S6K, MEK, ERK, p38, or JNK. Phase 2. Blood, 2015, 10.1182/blood-2014-11-610329 Clin Cancer Res, 2016, 10.1158/1078-0432. CCR-16-1971 Cell Rep, 2015, 11(3):446-59

Apitolisib (GDC-0980, RG7422) is a potent, class I PI3K inhibitor for PI3Kα/β/δ/γ with IC50 of 5 nM/27 nM/7 nM/14 nM in cell-free assays, respectively. Also a mTOR inhibitor with Ki of 17 nM in a cell-free assay, and highly selective versus other PIKK family kinases. Phase 2. Cancer Discov, 2014, 4(5):554-63 Breast Cancer Res, 2014, 16(4):406 Mol Cancer Ther, 2015, 14(8):1928-38

GSK1059615 is a dual inhibitor of PI3Kα/β/δ/γ (reversible) and mTOR with IC50 of 0.4 nM/0.6 nM/2 nM/5 nM and 12 nM, respectively. Phase 1. Nature, 2012, 486(7404):532-6 Nat Chem Biol, 2013, 9(11):708-14 Exp Mol Med, 2015, 47:e143

Gedatolisib (PF-05212384, PKI-587) is a highly potent dual inhibitor of PI3Kα, PI3Kγ and mTOR with IC50 of 0.4 nM, 5.4 nM and 1.6 nM in cell-free assays, respectively. Phase 2. Cell Rep, 2015, 11(3):446-59 Pigment Cell Melanoma Res, 2014, 10.1111/pcmr.12268 Mol Cancer Ther, 2015, 14(2):429-39

WYE-354 is a potent, specific and ATP-competitive inhibitor of mTOR with IC50 of 5 nM, blocks mTORC1/P-S6K(T389) and mTORC2/P-AKT(S473) not P-AKT(T308), selective for mTOR than PI3Kα (>100-fold) and PI3Kγ (>500-fold). Cancer Lett, 2015, 359(1):97-106 Exp Mol Med, 2015, 47:e143 Exp Mol Med, 2015, 47:e143

Vistusertib (AZD2014) is a novel mTOR inhibitor with IC50 of 2.8 nM in a cell-free assay; highly selective against multiple PI3K isoforms (α/β/γ/δ). AZD2014 showed no or weak binding to the majority of kinases when tested at 1 M.

These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

As it is presently understood, RNA interference involves a multi-step process. Double stranded RNAs are cleaved by the endonuclease Dicer to generate nucleotide fragments (siRNA). The siRNA duplex is resolved into 2 single stranded RNAs, one strand being incorporated into a protein-containing complex where it functions as guide RNA to direct cleavage of the target RNA (Schwarz et al, Mol. Cell. 10:537 548 (2002), Zamore et al, Cell 101:25 33 (2000)), thus silencing a specific genetic message (see also Zeng et al, Proc. Natl. Acad. Sci. 100:9779 (2003)).

The invention includes a method of treating CLEC16A related disorders such as obesity and neurological disease in a mammal. An exemplary method entails administering to the mammal a pharmaceutically effective amount of CLEC16A siRNA. The siRNA inhibits the expression of CLEC16A. Preferably, the mammal is a human. The term “patient” as used herein refers to a human.

Specific siRNA preparations directed at inhibiting the expression of CLEC16A, as well as delivery methods are provided as a novel therapy to treat obesity. SiRNA oligonucleotides directed to CLEC16A specifically hybridize with nucleic acids encoding CLEC16A and interfere with CLEC16A gene expression. The siRNA can be delivered to a patient in vivo either systemically or locally with carriers, as discussed below. The level of siRNA expressed can be controlled by methods known to those of skill in the art. The compositions of the invention may be used alone or in combination with other agents or genes encoding proteins to augment the efficacy of the compositions.

A “membrane permeant peptide sequence” refers to a peptide sequence which is able to facilitate penetration and entry of the CLEC16A inhibitor across the cell membrane. Exemplary peptides include without limitation, the signal sequence from Karposi fibroblast growth factor exemplified herein, the HIV tat peptide (Vives et al., J. Biol. Chem., 272:16010-16017, 1997), Nontoxic membrane translocation peptide from protamine (Park et al., FASEB J. 19(11):1555-7, 2005), CHARIOT® delivery reagent (Active Motif, U.S. Pat. No. 6,841,535) and the antimicrobial peptide Buforin 2.

In one embodiment of the invention siRNAs are delivered for therapeutic benefit. There are several ways to administer the siRNA of the invention to in vivo to treat obesity including, but not limited to, naked siRNA delivery, siRNA conjugation and delivery, liposome carrier-mediated delivery, polymer carrier delivery, nanoparticle compositions, plasmid-based methods, and the use of viruses.

siRNA composition of the invention can comprise a delivery vehicle, including liposomes, for administration to a subject, carriers and diluents and their salts, and/or can be present in pharmaceutically acceptable formulations. This can be necessary to allow the siRNA to cross the cell membrane and escape degradation. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192; Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule.

The frequency of administration of the siRNA to a patient will also vary depending on several factors including, but not limited to, the type and severity of the obesity or neurological disease to be treated, the route of administration, the age and overall health of the individual, the nature of the siRNA, and the like. It is contemplated that the frequency of administration of the siRNA to the patient may vary from about once every few months to about once a month, to about once a week, to about once per day, to about several times daily.

Pharmaceutical compositions that are useful in the methods of the invention may be administered systemically in parenteral, oral solid and liquid formulations, ophthalmic, suppository, aerosol, topical or other similar formulations. In addition to the appropriate siRNA, these pharmaceutical compositions may contain pharmaceutically-acceptable carriers and other ingredients known to enhance and facilitate drug administration. Thus, such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to administer the appropriate siRNA to a patient according to the methods of the invention. The use of nanoparticles to deliver siRNAs, as well as cell membrane permeable peptide carriers that can be used are described in Crombez et al., Biochemical Society Transactions v 35:p 44 (2007).

Methods of the invention directed to treating obesity involve the administration of CLEC16A siRNA in a pharmaceutical composition. CLEC16A siRNA is administered to an individual as a pharmaceutical composition comprising CLEC16A siRNA and a pharmaceutically acceptable carrier. Pharmaceutically acceptable carriers are well known in the art and include aqueous solutions such as physiologically buffered saline, other solvents or vehicles such as glycols, glycerol, oils such as olive oil or injectable organic esters.

A pharmaceutically acceptable carrier can contain physiologically acceptable compounds that act, for example, to stabilize the CLEC16A siRNA or increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the CLEC16A siRNA.

One skilled in the art appreciates that a pharmaceutical composition comprising CLEC16A siRNA can be administered to a subject by various routes including, for example, orally or parenterally, such as intravenously (i.v.), intramuscularly, subcutaneously, intraorbitally, intranasally, intracapsularly, intraperitoneally (i.p.), intracisternally, intra-tracheally (i.t.), or intra-articularly or by passive or facilitated absorption. The same routes of administration can be used other pharmaceutically useful compounds, for example, small molecules, nucleic acid molecules, peptides, antibodies and polypeptides as discussed hereinabove.

A pharmaceutical composition comprising CLEC16A siRNA inhibitor also can be incorporated, if desired, into liposomes, microspheres, microbubbles, or other polymer matrices (Gregoriadis, Liposome Technology, Vols. I to III, 2nd ed., CRC Press, Boca Raton Fla. (1993)).

Liposomes, for example, which consist of phospholipids or other lipids, are nontoxic, physiologically acceptable and metabolizable carriers that are relatively simple to make and administer. The pharmaceutical preparation comprises a siRNA targeting CLEC 16A or an expression vector encoding for an siRNA targeting CLEC 16A. Such pharmaceutical preparations can be administered to a patient for treating obesity or neurological diseases associated with aberrant CLEC16A function.

Expression vectors for the expression of siRNA molecules preferably employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502 09).

A formulated siRNA composition can be a composition comprising one or more siRNA molecules or a vector encoding one or more siRNA molecules independently or in combination with a cationic lipid, a neutral lipid, and/or a polyethyleneglycol-diacylglycerol (PEG-DAG) or PEG-cholesterol (PEG-Chol) conjugate. Non-limiting examples of expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500-505.

A lipid nanoparticle composition is a composition comprising one or more biologically active molecules independently or in combination with a cationic lipid, a neutral lipid, and/or a polyethyleneglycol-diacylglycerol (i.e., polyethyleneglycol diacylglycerol (PEG-DAG), PEG-cholesterol, or PEG-DMB) conjugate. In one embodiment, the biologically active molecule is encapsulated in the lipid nanoparticle as a result of the process of providing and aqueous solution comprising a biologically active molecule of the invention (i.e., siRNA), providing an organic solution comprising lipid nanoparticle, mixing the two solutions, incubating the solutions, dilution, ultrafiltration, resulting in concentrations suitable to produce nanoparticle compositions. Nucleic acid molecules can be administered to cells by incorporation into other vehicles, such as biodegradable polymers, hydrogels, cyclodextrins. (see for example Gonzalez et al., 1999, Bioconjugate Chem., 10, 1068-1074; Wang et al., International PCT publication Nos. WO 03/47518 and WO 03/46185), poly(lactic-co-glycolic) acid (PLGA) and PLCA microspheres (see for example U.S. Pat. No. 6,447,796 and US Patent Application Publication No. US 2002130430), biodegradable nanocapsules, and bioadhesive microspheres, or by proteinaceous vectors (O'Hare and Normand, International PCT Publication No. WO 00/53722).

Cationic lipids and polymers are two classes of non-viral siRNA delivery which can form complexes with negatively charged siRNA. The self-assembly PEG-ylated polycation polyethylenimine (PEI) has also been used to condense and protect siRNAs (Schiffelers et al., 2004, Nuc. Acids Res. 32: 141-110). The siRNA complex can be condensed into a nanoparticle to allow efficient uptake of the siRNA through endocytosis. Also, the nucleic acid-condensing property of protamine has been combined with specific antibodies to deliver siRNAs and can be used in the invention (Song et al., 2005, Nat. Biotech. 23:709-717).

In order to treat an individual having obesity or a neurological disease, to alleviate a sign or symptom of the disease, CLEC16A siRNA should be administered in an effective dose. The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. One skilled in the art would know that the amount of CLEC16A siRNA required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having obesity.

The effective dose of CLEC16A siRNA will depend on the mode of administration, and the weight of the individual being treated. The dosages described herein are generally those for an average adult but can be adjusted for the treatment of children. The dose will generally range from about 0.001 mg to about 1000 mg.

The concentration of CLEC16A siRNA in a particular formulation will depend on the mode and frequency of administration. A given daily dosage can be administered in a single dose or in multiple doses so long as the CLEC16A siRNA concentration in the formulation results in the desired daily dosage. One skilled in the art can adjust the amount of CLEC16A siRNA in the formulation to allow administration of a single dose or in multiple doses that provide the desired concentration of CLEC16A siRNA over a given period of time.

In an individual suffering from obesity, in particular a more severe form of the disease, administration of CLEC16A siRNA can be particularly useful when administered in combination, for example, with a conventional agent for treating such a disease. The skilled artisan would administer CLEC16A siRNA, alone or in combination and would monitor the effectiveness of such treatment using routine methods such as pulmonary function determination, radiologic, immunologic or, where indicated, histopathologic methods.

Administration of the pharmaceutical preparation is preferably in an “effective amount” this being sufficient to show benefit to the individual. This amount prevents, alleviates, abates, or otherwise reduces the severity of obesity symptoms in a patient.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

As mentioned previously, a preferred embodiment of the invention comprises delivery of the CLEC16A siRNA to a patient in need thereof, and candidate siRNA compositions for use in the invention are provided in Table 1. The sequences in Table 1 include several siRNA duplexes (i.e., sense and antisense sequences for a CLEC16A target region), as well as several sequences of ‘sense’ strand alone. Those of skill in the art can determine the sequence of an antisense siRNA strand based on the disclosure of the sense strand, and will appreciate the difference between “U” and “T” designations in the sequences which correspond to RNA and DNA molecules, respectively.

The following example is provided to illustrate certain embodiments of the invention. It is not intended to limit the invention in any way.

### Example I

Given the prominent role of CLEC16A SNP associations in variety of autoimmune disorders, we generated a novel whole-body Clec16a tamoxifen inducible knockout (KO) mouse, where tamoxifen treatment turns off CLEC16A expression in all organs at time points of interest (UBC-Cre-Clec6a^{loxP}). In our study, we discovered that turning off Clec16a in 8-10-week-old mice leads to severe weight loss (˜20%), robust inflammatory response and development of severe neurological symptoms, including ataxia. The mice exhibited a neuronal phenotype including tremors, impaired gait, and dystonic postures that worsen over time. Pathological analysis revealed that degenerating sensory axons, and Purkinje cell loss in the cerebellum account for this phenotype. Activated microglia and astrocytes were found in affected regions of the CNS. Affected and unaffected regions of the CNS and PNS showed increased levels of proteins related to impaired mitophagy and autophagy. These findings suggest that mitophagy and/or autophagy plays a role in certain types of spinocerebellar degeneration. Thus, ubiquitous inducible knockout of Clec16a in mice results in progressive neurodegeneration resembling spinocerebellar ataxia.

Our Clec16a KO mouse model shows mitochondrial defect and accumulation of unhealthy mitochondria. Our lab and others have shown a connection between Clec16a and autophagy in immunological and neurological cells (Redmann et al., 2016; Soleimanpour et al., 2014; Tam et al., 2017). We show that the extreme weight loss observed in the Clec16a KO mice is due to lipolysis (lipophagy) observed by the complete loss of fat and an increased phosphorylation of Hormone sensitive lipase (HSL) proteins in Western blots. Increased food intake observed in KO mice fails to rescue the white adipose atrophy. mRNA expression revealed upregulation of upregulation of catabolic and thermogenic genes together with downregulation of downstream adipogenic genes promoting HSL-mediated lipolysis in adipose tissue. Serum lipid analysis revealed significant decrease in Cholesterol, Triglycerides and free fatty acids and decreased adiponectin, leptin and upregulated LDL-Receptors in adipose tissue. Normal adipose tissue growth and function is critical to maintaining metabolic homeostasis and its excess (e.g. obesity) or absence (e.g. lipodystrophy) is associated with severe metabolic disease. In addition, elevated cytokine levels as measured by Proteome Profiler Mouse XL Cytokine Array, were observed concurrent with the lipolysis and could contribute to further wasting and the progressive neurodegeneration resembling spinocerebellar ataxia observed in the mice. Evidence for a link to SOCS proteins and the JAK/STAT pathway with autoimmune inflammatory phenotype and spinocerebral ataxia are presented respectively. Our whole body inducible Clec16a KO mouse, therefore provides a comprehensive murine model for use in future elucidation of mechanism and dug targets involved in healthy form of weight loss, Autoimmune-inflammatory phenotype and spinocerebellar degeneration.

Clec16a KO of >60% in UBC-Cre-Clec16a^{loxP} Mice Exhibit Adipose Tissue Atrophy and Severe Weight Loss.

We employed UBC-Cre-Clec16a^{loxP} mice—an inducible KO model to study CLEC16A's role in autoimmunity. We choose this model to circumvent possible embryonic lethality and determine the effect of CLEC16A loss in adult mice.

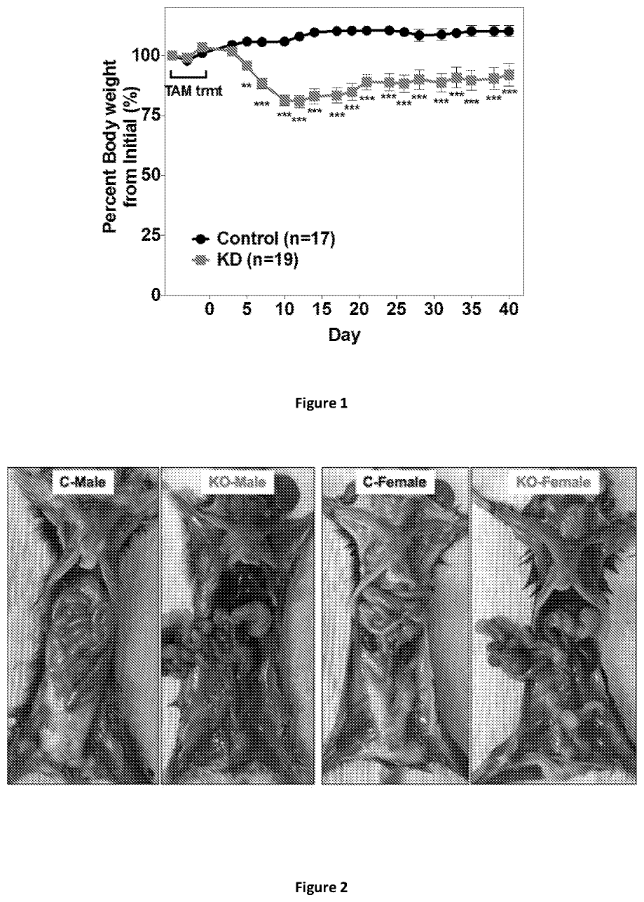

The first visible observation we made in control and Clec16a KO mice, fed on regular chow diet was difference in body weight. Clec16a knockout mice exhibit severe weight loss starting 1 week after initiation of tamoxifen treatment in comparison to control mice. During the same time period, control mice showed a healthy appearance and maintained their body weight throughout the study in comparison to Clec16a KO (FIG. 1). All mice with greater than 60% of Clec16a KO exhibited significant reduction of body weight which deteriorated over the length of the study with increased severity in neurological symptoms, and an elevated morbidity rate (FIG. 1). According to previously reports, gender differences do exist for T1D pathogenesis, MS, and other autoimmune disorders. The disease incidence lays around 60-80% in females and 20-30% in males. For the purpose of healthy examination and to determine the cause of weight loss, the Clec16a KO and control mice were dissected.

Compared to control mice, both male and female Clec16a KO mice exhibited near to complete absence of typical gonadal adipose fat tissue (FIG. 2). Further examination indicated that all the white adipose tissue (WAT) depots, including gonadal, inguinal, mesenteric, retroperitoneal, perineal, and pericardial, were remarkably reduced or absent in Clec16a KO mice (FIG. 2). Thus, Clec16a loss promotes body weight reduction and fat loss leading to lipodystrophy of adipose tissue, a phenotype similar to that observed in mammalian models with lipodystrophy and has profound effect on WAT deposits in adult mice. These results suggest that Clec16a KO modulates lipid metabolism and triggers abnormal fat loss possibly mediated by lipolysis (lipophagy).

UBC-Cre-Clec16a^{loxP} KO Mice Exhibit Increased Food Intake.

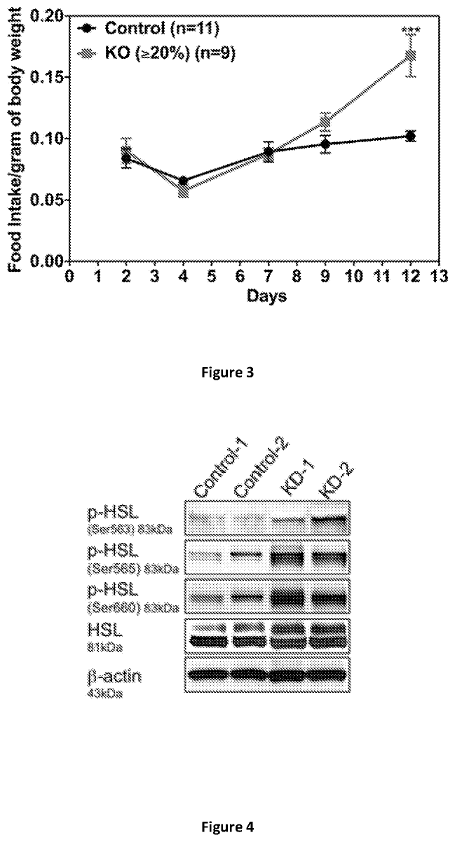

We performed food intake study to rule out the amount of food consumed as a possible reason behind the severe weight loss. Clec16a KO mice consumed as much or more food in comparison to the control mice. Thus, less food consumption is not the reason behind the weight loss of Clec16a KO mice (FIG. 3). Despite increased food consumption Clec16a KO mice kept losing weight rapidly. These results suggest that CLEC16A modulates fat loss either by reduced efficiency in storage or an increased energy expenditure, or both in the Clec16a KO mice.

Clec16a KO Leads to Abnormal Fat Loss by Accelerated Lipolysis in Adipose Tissues of UBC-Cre-Clec16aloxP KO Mice.

To examine the signaling underlying the fat loss in Clec16a KO we used immunoblot analysis to assess the role of Clec16a in inducing lipolysis (lipophagy). HSL (hormone-sensitive lipase) is a key enzyme in the mobilization of fatty acids in adipocytes as well as non-adipocytes. Triacylglycerol is stored in lipid droplets as a primary energy reserve. During lipolysis, triacylglycerols in adipocytes are hydrolyzed into free fatty acids and glycerol. Phosphorylation of HSL at Ser563, Ser659, and Ser660 by PKA stimulates HSL activity, which in turn catalyzes the hydrolysis of triacylglycerol. We found increased phosphorylation of HSL (FIG. 4). Our results indicate that CLEC16A functions to restrain lipolysis in adipose tissues and loss of CLEC16A triggers lipolysis.

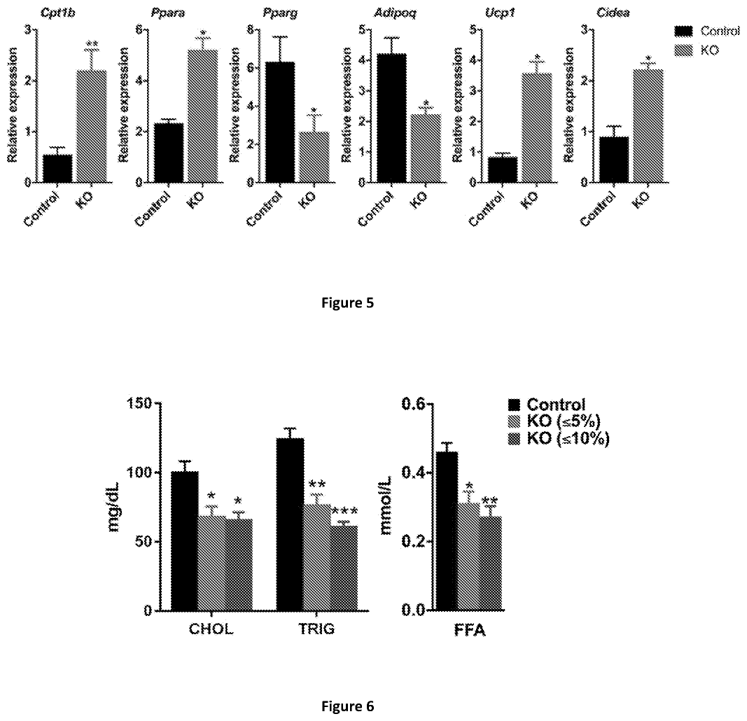

To gain insight in the mechanism(s) whereby Clec16a mediates its effect on energy expenditure to induce fat loss, we measured expression of key genes regulating lipid metabolism in gonadal white adipose tissue (gWAT) of Clec16a KO mice. We found that carnitine palmitoyltransferase 1b (Cpt1b), a gene essential for adipose tissue fatty acid oxidation, was significantly upregulated in Clec16a KO gWAT, along with the upstream transcription factor, peroxisome proliferator-activated receptor alpha (Ppara). Further, the expression of the adipogenic gene, Pparg and its downstream target adiponectin precursor (Adipoq) were significantly reduced in Clec16a KO gWAT. Thermogenic genes, thermogenin (Ucp1) and cell death inducing DFFA-like effector A (Cidea) were significantly upregulated in WAT of Clec16a mice (FIG. 5). Thus, fat loss in Clec16a KO mouse is mediated through upregulation of catabolic and thermogenic genes together with downregulation of downstream adipogenic genes promoting HSL-mediated lipolysis. Increased food intake in KO mice fails to rescue the white adipose atrophy.

Lipolysis is defined as the catabolism of triacylglycerols stored in cellular lipid droplets. New findings that lipolytic products and intermediates participate in cellular signaling processes and is particularly important in many non-adipose tissues unveils a previously underappreciated aspect of lipolysis, which may be relevant for human diseases. Normal adipose tissue growth and function is critical to maintaining metabolic homeostasis and its excess (e.g. obesity) or absence (e.g. lipodystrophy) is associated with severe metabolic disease. Decreased triglyceride storage leads to adipocyte lipotoxicity, mitochondrial dysfunction and increased oxidative stress. This results in production of inflammatory mediators and deregulated release of free fatty acids. This contributes to impaired insulin sensitivity and adverted liver, muscles and heart functions leading to early complications.

Serum Lipid Analysis of Clec16a KO and Control Mice.

We also performed lipid analysis on serum of control and Clec16a KO (score 2 and 4) mice. A significant decrease in cholesterol, triglycerides and free fatty acid was observed in the serum of Clec16a KO mice compared to control (FIG. 6). These results suggest that, controlled Clec16a expression could be of therapeutic potential in promoting a healthier form of weight loss.

UBC-Cre-ER^{T2}-Clec16a^{loxP/loxP} KO Mice Exhibit Increased Cytokine Levels in Adipose Tissue and Plasma.

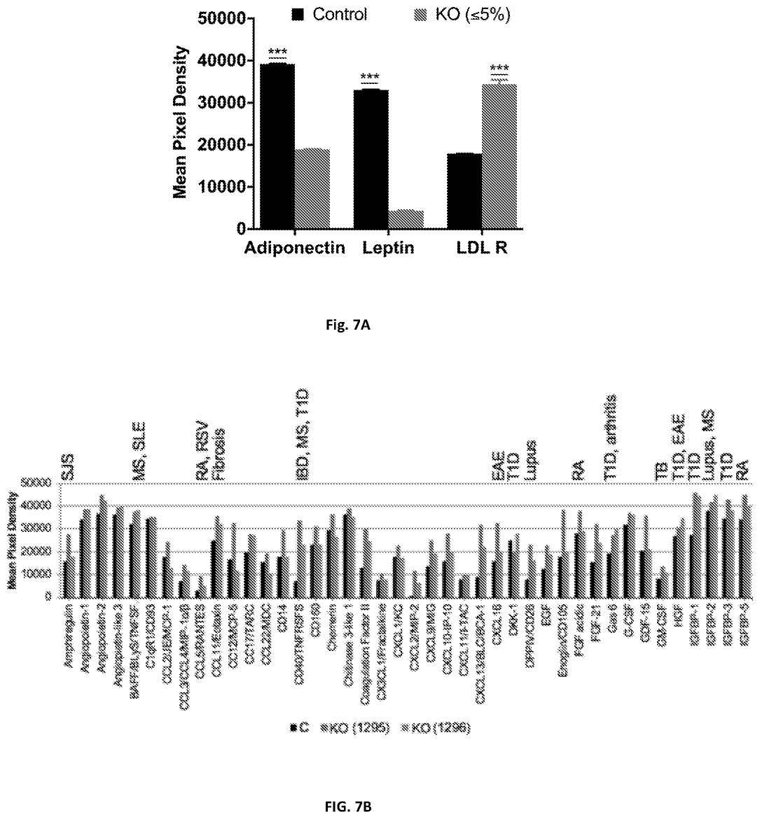

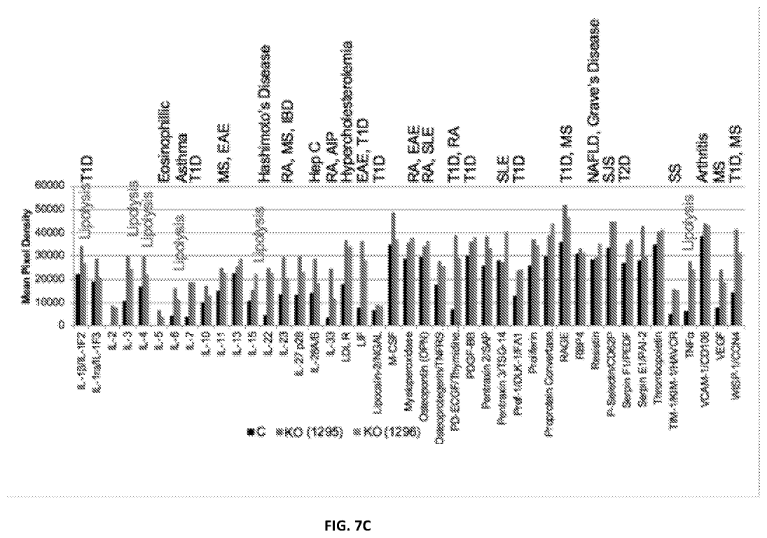

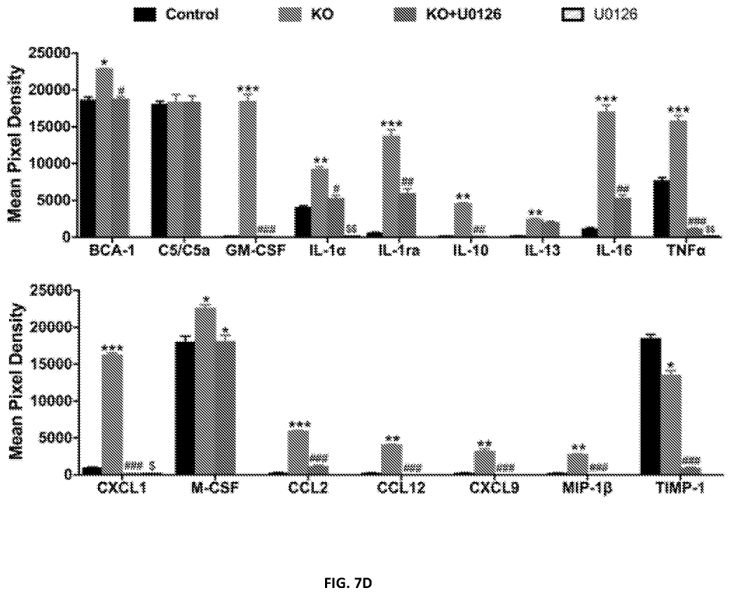

To gain insight how Clec16a KO, weight loss and lipolysis promote a dynamic immune response in murine adipose tissue and may contribute to disease pathogenesis, we evaluated adipose tissue and plasma of control and Clec16a KO mice in the Proteome Profiler Mouse XL Cytokine Array. The Proteome Profiler Mouse XL Cytokine Array Kit is a membrane-based sandwich immunoassay allows parallel determination of the relative levels of selected mouse cytokines and chemokines. Adiponectin and leptin from adipose tissue play a key role in energy homeostasis and metabolism. Clec16a KO mice exhibit decreased adiponectin, leptin and LDL-R compared to control (FIG. 7a). In addition, we saw up-regulation of several cytokine, chemokines and inflammatory markers in adipose tissue of Clec16a KO mice that have been associated with several autoimmune disorders (FIG. 7b, c). Our results indicate that the signal originates from dysregulated lipolysis (adipose tissue) and promotes wasting.

To gain insight in the inflammatory mechanism involved in the development, progression and pathogenesis of various autoimmune diseases, we profiled plasma from control, KO and U0126-treated KO mice for cytokines and chemokine using Mouse Cytokine Array panel. Plasma from Clec16a KO mice showed upregulation of Th1 cytokines (TNF-α, IL-1, & IL-16), vs. low levels of Th2 (IL-10 & IL-13) and elevated levels key chemokines GM-CSF, KC (CXCL1) JE (MCP-1), MCP-5, MIG (CXCL9), MIP-1b (CCL4) in comparison to control (FIG. 7d).

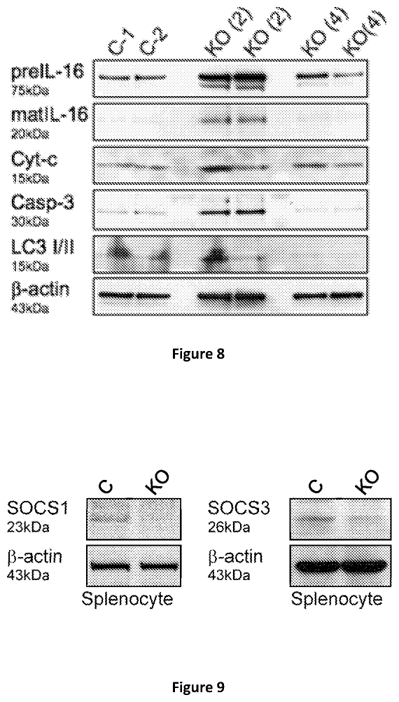

U0126 inhibitor treatment reversed all the up regulated cytokines and chemokines, suggesting that the inflammatory mechanism involved with autoimmune risk is mediated by dysregulated mitophagy and can be corrected by mitophagy inhibitors. Our results provide critical evidence in support for role of dysregulated lipolysis and Clec16a loss leads to progression of autoimmunity as depicted in graph (FIG. 7b-d). Increase in IL-16 in the plasma cytokine levels could contribute to the neurological degeneration seen in the Clec16a KO mice (FIG. 7d). Our Immunoblot analysis indicates constitutively high expression of IL-16 precursor and bioactive IL-16 in Clec16a KO splenocyte further support the role of cytokine mediated neurodegeneration (FIG. 8). The cytokine IL-16 is a CD4⁺T cell-specific chemoattractant that is biased towards CD4+Th1 cells. IL-16 precursor is constitutively expressed in lymphocytes and during CD4⁺T cell activation; active caspase-3 cleaves and releases C-terminal bioactive IL-16. The connection between increased cytokines and neurodegeneration is known (Khaibullin et al., 2017). It is also known that the presence of CLEC16A MS risk alleles correlate with reduced SOCS1 and DEXI expression in the thymus through a regulatory element (Leikfoss et al., 2013).

SOCS Protein Expression is Decreased in UBC-Cre-Clec16a^{loxP} KO Mice.

Based on the observed loss of visceral and subcutaneous fat, and food intake study, our ubiquitous Clec16a KO mice display a phenotype similar to that observed in lipodystrophy. Dysregulated lipolysis contributes to lipotoxicity, mitochondrial dysfunction and increased oxidative stress resulting in production of inflammatory mediators. CLEC16's genomic location next to the suppressor of cytokine signaling 1 (SOCS1) gene and the expression specificity in immune cells including dendritic cells, B & T-lymphocytes and natural killer (NK) cells, which are pivotal in the pathogenesis of several autoimmune disorders, led us to hypothesize that CLEC16A exerts its effect on a wide variety of immune cells via modulating SOCS expression and regulating cytokine signaling. The SOCS (suppressor or cytokine signaling) family members are negative regulators of cytokine signal transduction that inhibit the Jak/Stat pathway. These proteins are important regulators of cytokine signaling, proliferation, differentiation, and immune responses and are involved in regulating over 30 cytokines, including interleukins, growth hormone (GH), interferon, leptin, and leukemia inhibitory factor. SOCS1 shares the most homology with SOCS3 and both are highly induced by cytokines. Both SOCS1 and SOCS3 directly inhibit Jak activity. Jak (Janus Kinase) and Stat (signal transducer and activator of transcription) proteins are play important roles in inflammatory immune responses (Fenner et al., 2006), and therefore, regulation of Jak/Stat signaling is crucial to prevent aberrant signaling which can lead to disease progression.

To examine the mechanism involved behind the inflammatory cytokine storm, we examined the levels of SOCS1 and SOCS3 expression in an immunoblot analysis. Splenocytes from Clec16a KO exhibit decreased expression of SOCS1 and SOCS3 compared to control (FIG. 9). With SOCS protein levels decreased, cytokine production is not suppressed resulting in the increased cytokine levels contributing to inflammation, concomitant lipolysis and neurodegeneration observed in the Clec16a KO mice. In light of our data, and given the association of Clec16a with several autoimmune disorders, it appears that the molecular link between CLEC16A, lipophagy, and SOCS is abnormal and leads to autoimmune disorders.

The Pan JAK Inhibitor Tofacitinib Suppresses SOCS1-JAK-STAT Mediated Cytokine Signaling and Improves Survival of Clec16a KO Mice.

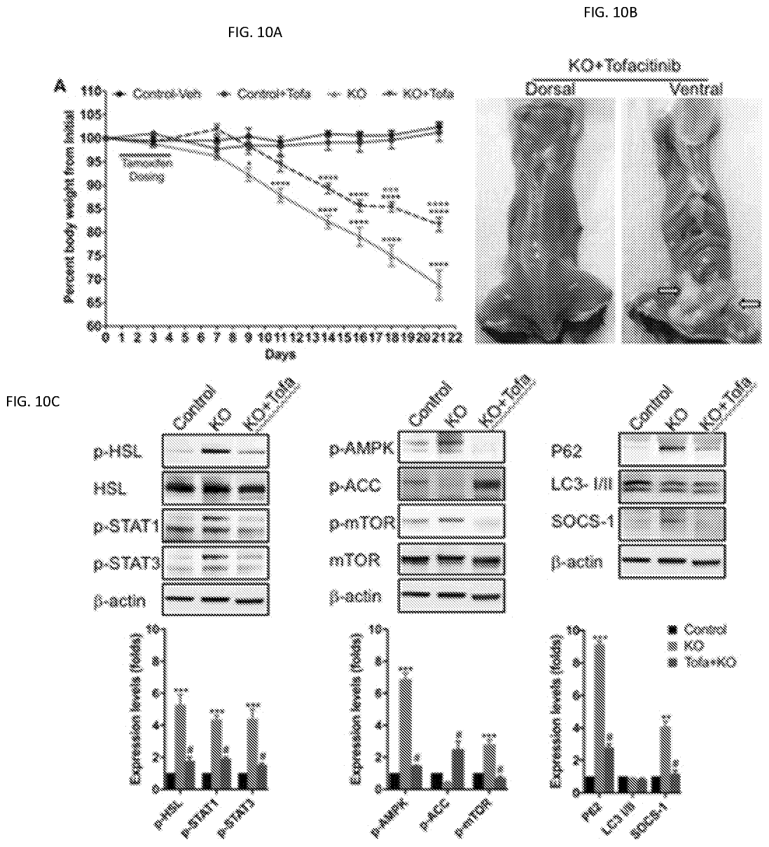

In light of the above findings and established CLEC16A association with several autoimmune disorders, we hypothesized that upregulated JAK/STAT signaling observed in CLEC16A KO mice could be rescued using a JAK/STAT inhibitor. Tofacitinib treatment significantly rescued the fat and weight loss in CLEC16A KO mice and improved the survival curve (FIG. 10).

To examine the signaling mechanism underlying the rescue, we performed immunoblot analysis on gWAT isolated from control, KO and Tofacitinib treated mice. We evaluated HSL, STATs, AMPK, mTOR, P62, LC3I/II and SOCS-1 expression (FIG. 10C). Tofacitinib treatment significantly reversed the SOCS1 expression observed in CLEC16A KO. Immunoblot analysis revealed significant upregulation of phospho-HSL in CLEC16AKO and significant reversal of p-HSL comparable to normal in CLEC16A KO+Tofacitinib mice. CLEC16A KO mice adipose further exhibits significant upregulation of p-STAT1 and p-STAT3. Tofacitinib rescued the inflammatory phenotype by downregulating the p-STAT1 and p-STAT3 to normal levels comparable to control. We observed significantly enhanced phosphorylation of AMPK. However, its target ACC exhibited reduced phosphorylation in CLEC16A KO. Tofacitnib treatment significantly reduced the upregulated p-AMPK and promoted the phosphorylation of ACC. Another downstream effector of AMPK is mTOR signaling that controls a plethora of functions and is a regulator of autophagy. We observed significant increase in phosphorylation of mTOR and reversal in tofacitinib treated CLEC16A KO mice correcting the autophagy defect. Over-activation of mTOR indicates inhibition of autophagy/lipophagy as evident by significant accumulation of P62 in CLEC16A KO mice. Tofacitinib treatment also rescues the autophagy/lipophagy defect (FIG. 10C). Thus, Tofacitinib exerts its multifaceted effect on AMPK, mTOR, HSL, STATs and autophagy/lipophagy signaling and rescues the lipodystrophic inflammatory phenotype exhibited by CLEC16A KO mice.

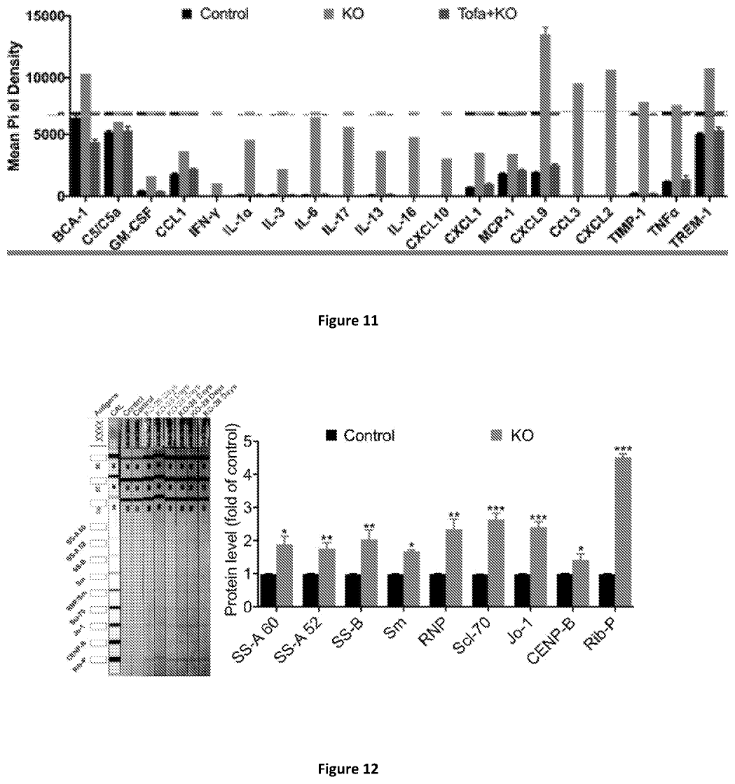

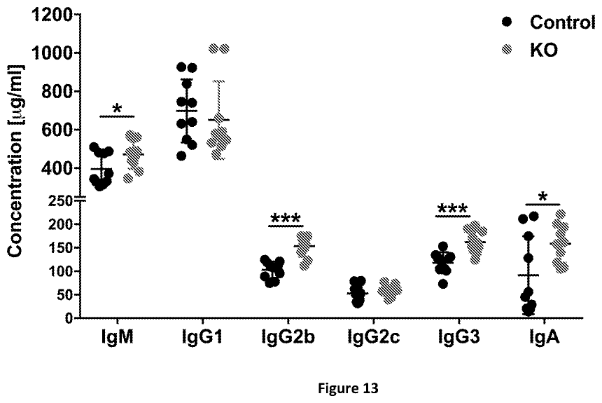

Increased cytokines/chemokine levels reflect upon the inflammatory mechanism utilized during the development, progression and pathogenesis of various autoimmune and inflammatory diseases. Our results indicate that CLEC16A knockout inflammatory phenotype is attenuated by Tofacitinib (FIG. 11).

CLEC16A KO Induces Susceptibility to Autoimmunity in Mice.

We used C57Bl/6 mice, to test the hypothesis that altered CLEC16A expression can induce autoimmune responses in a genetic background that does not spontaneously express an autoimmune phenotype (Hudson et al., 2003). This model can therefore be used not only to trace the pathogenesis of the autoimmune responses, but also to explore how CLEC16A KO might trigger the autoimmune response through modified immune regulation.

CLEC16A KO-Induced Autoantibodies.

Serum samples from Control and CLEC16A KO mice were assayed for antibodies to various nuclear antigens using a line assay Western blot. ANA-9-Line Immunoblot assay is a membrane-based enzyme immunoassay for the semi-quantitative measurement of IgG class autoantibodies to extractable nuclear antigens SS-A 52, SS-A 60, SS-B, RNP/Sm, Sm, centromere B, Jo-1, Scl-70 and ribosomal P proteins in serum or plasma. These results show that CLEC16A KO led to production of antinuclear antibodies indicative of systemic autoimmune disease.

Our finding of upregulated specific antibodies in the sera from CLEC16A KO mice is interesting as these antibodies are also found in SLE and other systemic autoimmune diseases. Further characterization of the specific target autoantibodies in this model is needed, as this may provide clues regarding the mechanisms of lost tolerance to self-antigens.

Serum Immunoglobulin Isotyping.

In order to determine whether CLEC16A KO led to changes in serum immunoglobulin isotypes, isotypes and the IgG subclasses were measured sera. IgM and IgA showed significant upregulation in CLEC16A KO mice sera compared to control. IgG subclasses IgG1, IgG2b, and IgG3 were statistically significant. IgG2c showed no change. IgG2b, the predominant subclass in sera of C57B16 mice showed upregulation in CLEC16A KO mice. This antibody subclass actively binds complement and therefore can be considered potentially pathogenic leading to Th1 phenotype. Another IgG subclass IgG3, potent proinflammatory antibody despite its shorter half-life and effective in induction of effector functions showed significant upregulation in CLEC16A KO. Serum Immunoglobulin Isotyping results are indicate of excessive inflammatory response.

Ubiquitous Inducible Knockout of CLEC16A in Mice Results in Progressive Neurodegeneration Resembling Spinocerebellar Ataxia.

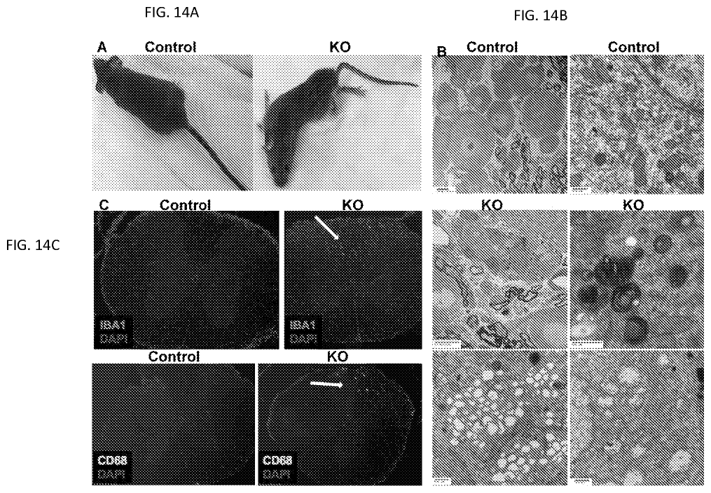

Our whole body inducible CLEC16A KO mice exhibits a neuronal phenotype including tremors, impaired gait, and dystonic postures that worsen over time (FIG. 14).

Pathological analysis revealed that degenerating sensory axons, and Purkinje cell loss in the cerebellum account for this phenotype. Activated microglia and astrocytes were found in affected regions of the CNS. Affected and unaffected regions of the CNS and PNS showed increased levels of proteins related to mitophagy and autophagy. These findings suggest that mitophagy and/or autophagy might play a role in some kinds of spinocerebellar degeneration. The selective involvement of cerebellar and primary sensory neurons models a human disease known as spinocerebellar ataxia, which has diverse genetic causes (Huang and Verbeek, 2018). Our whole body inducible Clec16a KO mouse, therefore provides a comprehensive murine model for use in future elucidation of mechanism and dug targets involved in healthy form of weight loss, Autoimmune-inflammatory phenotype and spinocerebellar ataxia.

### CONCLUSION

Our results underscore critical role of CLEC16A action in immune cells and indicate that a delicate balance of CLEC16A activity appears to be needed for cellular homeostasis. In our study, we discovered that turning off CLEC16A in 8-10-week-old mice leads to severe weight loss (˜20%), robust inflammatory response and development of severe neurological symptoms, including ataxia with progressive neurodegeneration resembling spinocerebellar ataxia. In, patient populations harboring variants that result in CLEC16A hypofunction, drugs with modulatory effects on mitophagy/autophagy/SOCS1 signaling could compensate for the attenuated CLEC16A activity and present formidable candidates for targeted interventions.

### Example II

### siRNA Compositions and Methods for Partial Inhibition of CLEC16A Expression

Based on these data, partial blockage of CLEC16A (or any of its pathway members) should result in weight loss which will benefit patients with obesity, without impacting susceptibility for autoimmune or neurological conditions.

As discussed above, one way to achieve partial reduction of CLEC16A expression levels is to introduce CLEC16A directed siRNAs into cells. A series of RNAs targeting CLEC16A are provided herein below.

TABLE 1
Candidate siRNA constructs for CLEC16A
Corresponding GenBank Accession number: NM_015226

| | |
|---|---|
| SEQ ID NO: 1 | CAAAAAAAUGUCUUCUCCCtt |
| SEQ ID NO: 2 | AAAUAUCCAAAGGAGAUGUtt |
| SEQ ID NO: 3 | CUGCUAUAGCAGAAAACCAtt |
| SEQ ID NO: 4 | UUCUCUUUUAUUGCCAAGUtt |
| SEQ ID NO: 5 | GAGGUUUUCUAACCCUCGGtt |
| SEQ ID NO: 6 | CAAAACGUGGUACAGAUACtt |
| SEQ ID NO: 7 | UUCUGUGACUGUGGUGUUUtt |
| SEQ ID NO: 8 | CUAGCAGGUUCCGGUUCUGtt |
| SEQ ID NO: 9 | CUCCACUAGCAGGUUCCGGtt |
| SEQ ID NO: 10 | GAUGGUCUCCACUAGCAGGtt |
| SEQ ID NO: 11 | CAAGAAGAAAACAAACAUAtt |
| SEQ ID NO: 12 | ACACGUAACGGCCCGACUUtt |
| SEQ ID NO: 13 | UGAGGUCUCGUGACUGAUGtt |
| SEQ ID NO: 14 | CUCAUCAGAAAAGUCAAAUtt |
| SEQ ID NO: 15 | GAGUUUUAACGAAAGUGUU~~ |
| SEQ ID NO: 16 | AAAGAAAUGGACAGUGUGGtt |
| SEQ ID NO: 17 | UGUGUACAGGGCAAAGUCAtt |
| SEQ ID NO: 18 | GGUUGAAAAACUUGAUGGCtt |
| SEQ ID NO: 19 | UUCAGGGUGGUUGAAAAACtt |
| SEQ ID NO: 20 | ACUUUAUAGACAUUCAAAGtt |
| SEQ ID NO: 21 | CCCAAUGAACCAGACCAAAtt |
| SEQ ID NO: 22 | UCUGCACGCAGUCAUCGAGtt |
| SEQ ID NO: 23 | GUUGAUGAUCAGGAUGUCAtt |
| SEQ ID NO: 24 | AUCGUUGAGGAACUCACAGtt |
| SEQ ID NO: 25 | GUGGUCAGUGAGCACAUCGtt |
| SEQ ID NO: 26 | AGCGGUGCAUGAUGUAUAAtt |
| SEQ ID NO: 27 | AAUGACUUCAGCUAACGAGtt |
| SEQ ID NO: 28 | GAUCACCAUUCAGAAUGACtt |
| SEQ ID NO: 29 | CAUCUCAGACAGAUCACCAtt |
| SEQ ID NO: 30 | CUGAAUAUCCUGUUCAGUCtt |
| SEQ ID NO: 31 | AACUUCUCUGAAUAUCCUGtt |
| SEQ ID NO: 32 | CCUCUUGCCCUUGUGCUUGtt |
| SEQ ID NO: 33 | UGGGCCCUUUCUCCUCAUCtt |
| SEQ ID NO: 34 | GGCAUCCUCGGUGGGCCCUtt |
| SEQ ID NO: 35 | GCUGGAUUCGCUCUAAUUUtt |
| SEQ ID NO: 36 | AUCUGGCUGGGCAGCGUUGtt |
| SEQ ID NO: 37 | CAGCGUCGCCAGCCGGAUCtt |
| SEQ ID NO: 38 | ACUCAUCAGGACUUGCUGCtt |
| SEQ ID NO: 39 | AGCCAGCACUCAUCAGGACtt |
| SEQ ID NO: 40 | GCAGGCCAGGUGCACGuCCtt |
| SEQ ID NO: 41 | ACAUGUCCAAAAAAAUGUC~~ |
| SEQ ID NO: 42 | UCAUGCUCCUAUACUCAUCtt |
| SEQ ID NO: 43 | AGGCUUGUGGAUGGUGAUGtt |
| SEQ ID NO: 44 | CUGGUCAGGAAGGUGAGGCtt |
| SEQ ID NO: 45 | UGCGCUCCUGGCCACGUUCtt |
| SEQ ID NO: 46 | GGCUGCGCUCCUGGCCACGtt |
| SEQ ID NO: 47 | UGGGUGCUCCCAGUGUGUCtt |
| SEQ ID NO: 48 | GGAGCGCAGGGAUAGGUGGtt |
| SEQ ID NO: 49 | UGAUGUUUUUGGACUUCUUtt |
| SEQ ID NO: 50 | GUUGAUGUUUUUGGACUUCtt |
| SEQ ID NO: 51 | AGUGUUGAUGUUUUUGGACtt |
| SEQ ID NO: 52 | AACCUUAGUGUUGAUGUUUtt |
| SEQ ID NO: 53 | CAAACCUUAGUGUUGAUGUtt |
| SEQ ID NO: 54 | ACUUCAUUAUUCCACAGGCtt |
| SEQ ID NO: 55 | UUUCCAGGAAGGUACUUCAtt |
| SEQ ID NO: 56 | GUAUCUCCCUUGUUUUUGU~~ |
| SEQ ID NO: 57 | GGAAGCUGUCUCUGUUUGGtt |
| SEQ ID NO: 58 | UCUUCACGGUCCCAACUGGtt |
| SEQ ID NO: 59 | GGCCACAGGGUCGGGAGUCtt |
| SEQ ID NO: 60 | GUCUGAUGCAUCUGGGUCCtt |
| SEQ ID NO: 61 | CCGAGUAGAAAGGAACAUCtt |
| SEQ ID NO: 62 | UGGCAGGUCUGGAGGAAAGtt |
| SEQ ID NO: 63 | GGAGACAGAGACCAUGUUCtt |
| SEQ ID NO: 64 | CGAGGAGACAGAGACCAUGtt |
| SEQ ID NO: 65 | AAACUGCCCUCGGCAGGCCtt |
| SEQ ID NO: 66 | UUGGCAGGACUGUGUUUCCtt |
| SEQ ID NO: 67 | CUCCUUGGCAGGACUGUGU~~ |
| SEQ ID NO: 68 | UGGGCGCCACUCCCCCUCCtt |
| SEQ ID NO: 69 | CCUUUCUGAGCUGUGCGUU~~ |
| SEQ ID NO: 70 | GUUUGUCCUUCCUUUGGGUtt |
| SEQ ID NO: 71 | CUUGGGGUGGGGCAGCCACtt |
| SEQ ID NO: 72 | CUGGGUGAGGUGGGGUCCUtt |
| SEQ ID NO: 73 | ACAAAAAUUAUUUACAUAUtt |
| SEQ ID NO: 74 | UCACUGGGACAAAAAUUAUtt |
| SEQ ID NO: 75 | GUUCUCACUGGGACAAAAAtt |
| SEQ ID NO: 76 | GCUCAGAGGCAUCGAGGUUtt |
| SEQ ID NO: 77 | AGGCUCAGAGGCAUCGAGGtt |
| SEQ ID NO: 78 | UCACUCAGUUUACCCCGAtt |
| SEQ ID NO: 79 | GGAGGAACCGCGAGCCGCCtt |
| SEQ ID NO: 80 | GUGGAUGUUGCGGGAAGUCtt |
| SEQ ID NO: 81 | GUGGUCCAAGGAGUGGAUGtt |
| SEQ ID NO: 82 | UGUUCUGUGACUGUGGUGUtt |
| SEQ ID NO: 83 | CAAAUACAGAGCUGUCAUUtt |
| SEQ ID NO: 84 | GUCAAAUACAGAGCUGUCAtt |
| SEQ ID NO: 85 | GAAGAAAACAAACAUAUUCtt |
| SEQ ID NO: 86 | CGACUUUUGCCGCAAGAUGtt |
| SEQ ID NO: 87 | GCACACGUAACGGCCCGACtt |
| SEQ ID NO: 88 | GAUGUUCUCAAAGAGGAUGtt |
| SEQ ID NO: 89 | AUAGAAUUUACGUAGUUAUtt |
| SEQ ID NO: 90 | GAUGAUAGAAUUUACGUAG~~ |
| SEQ ID NO: 91 | UUAUGAACGAUGAUAGAAUtt |
| SEQ ID NO: 92 | UUGAGUUUUAACGAAAGUG~~ |
| SEQ ID NO: 93 | GGACAGUGUGGUUGUUGAGtt |
| SEQ ID NO: 94 | GAAAUGGACAGUGUGGUUGtt |
| SEQ ID NO: 95 | AAAGUCAUUGGUGUGCUCAtt |
| SEQ ID NO: 96 | AACCAUGCUUUCAGGGUGG~~ |
| SEQ ID NO: 97 | CAGCAAUUCUAACCAUGCUtt |
| SEQ ID NO: 98 | GUUAUGGUUCUUACAGCAAtt |
| SEQ ID NO: 99 | ACAUUCAAAGUUAUGGUUCtt |
| SEQ ID NO: 100 | UAGACAUUCAAAGUUAUGGtt |
| SEQ ID NO: 101 | CAAUGACACUUUAUAGACAtt |
| SEQ ID NO: 102 | CUGGUUAUCCAAUGACACUtt |
| SEQ ID NO: 103 | GUAGUGCAGCAUGGCCUGG~~ |
| SEQ ID NO: 104 | GAAGUAAGGAACAGCAGUUtt |
| SEQ ID NO: 105 | GAGAAGUAAGGAACAGCAGtt |
| SEQ ID NO: 106 | UGCCACCAGAUCACUCAGUtt |
| SEQ ID NO: 107 | GAGGGGCAGGAAGAGCCUGtt |
| SEQ ID NO: 108 | UUCUCCUCCCUUGUCCUGGtt |
| SEQ ID NO: 109 | UUUCGGCCGUUCUCCUCCCtt |
| SEQ ID NO: 110 | GCAGGCUAAUUUUCGGCCG~~ |
| SEQ ID NO: 111 | AGACACCGGCAGGCUAAUU~~ |
| SEQ ID NO: 112 | AGAGACACCGGCAGGCUAAtt |
| SEQ ID NO: 113 | AUGCUGGGCUUGGCAGAACtt |
| SEQ ID NO: 114 | GAAGCACCGAAUGCUGGGCtt |
| SEQ ID NO: 115 | CUCGAGUGUCUCGGUGGGUtt |
| SEQ ID NO: 116 | CCGCCUCUUGCCCUUGUGCtt |
| SEQ ID NO: 117 | UUGCACCCGCCUCUUGCCCtt |
| SEQ ID NO: 118 | UCUCUUUUGCACCCGCCUCtt |
| SEQ ID NO: 119 | UUUUGUAGUUGGGUCUCUUtt |
| SEQ ID NO: 120 | GUUUUUGUAGUUGGGUCUCtt |
| SEQ ID NO: 121 | UUCCCCAACGUUUUUGUAGtt |
| SEQ ID NO: 122 | UUCUUCUUCCCCAACGUUUtt |
| SEQ ID NO: 123 | UCUUCUUCUUCCCCAACGUtt |
| SEQ ID NO: 124 | CUUUCUCCUCAUCUUCUUCtt |
| SEQ ID NO: 125 | GCCCUUUCUCCUCAUCUUCtt |
| SEQ ID NO: 126 | UAGCCUUCUCGGCGUCUUCtt |
| SEQ ID NO: 127 | CUUUAGCCUUCUCGGCGUCtt |
| SEQ ID NO: 128 | ACCCUCUGUACCUUUAGCCtt |
| SEQ ID NO: 129 | UGAACCACCCUCUGUACCUtt |
| SEQ ID NO: 130 | CCACUCGUCUUGAUGCCUUtt |
| SEQ ID NO: 131 | CCCCACUCGUCUUGAUGCCtt |
| SEQ ID NO: 132 | UUCACUCUCCCCACUCGUCtt |
| SEQ ID NO: 133 | UGAUCACCAUCUCGAUCUCtt |
| SEQ ID NO: 134 | GGCGGCCAGCUCUGAGAGCtt |
| SEQ ID NO: 135 | UUUCUCCUCGUCCGUGGUGtt |
| SEQ ID NO: 136 | GCAGGUGGCGGCGGCGCUUtt |
| SEQ ID NO: 137 | GAGCAGGUGGCGGCGGCGCtt |
| SEQ ID NO: 138 | CCAGGAAGGGUCUGCUCCAtt |
| SEQ ID NO: 139 | UUCAGGAUCCAUGCCUUUAtt |
| SEQ ID NO: 140 | UUUUUCAGGAUCCAUGCCU~~ |
| SEQ ID NO: 141 | GAGCUGGAUUCGCUCUAAUtt |
| SEQ ID NO: 142 | UUUGGCACGGGGAGCUGGAtt |
| SEQ ID NO: 143 | GUGGUCUUCUCGGCCGCAUtt |
| SEQ ID NO: 144 | CGGGUGGUUGUAGGUGGUCtt |
| SEQ ID NO: 145 | UCUUUCAGCUAGCGGGUGGtt |
| SEQ ID NO: 146 | UCAUGAUCCUGAUGAGUCUtt |
| SEQ ID NO: 147 | GUACAAGGUGAACACUUUCtt |
| SEQ ID NO: 148 | GUCGUACAAGGUGAACACU~~ |
| SEQ ID NO: 149 | UCCACGUUCAUGGGCUUCAtt |
| SEQ ID NO: 150 | AUAUUCCACGUUCAUGGGCtt |
| SEQ ID NO: 151 | CAUCAUGAGAUAUUCCACGtt |
| SEQ ID NO: 152 | AGGCGUCCAUCAUGAGAUA~~ |
| SEQ ID NO: 153 | CCCGUCAGUGGCGUGCCUGtt |
| SEQ ID NO: 154 | AUCGCCACACGGCAGCCGCtt |
| SEQ ID NO: 155 | CCGGAUGGCCCGCCGGGUCtt |
| SEQ ID NO: 156 | UCUCAGGCUCCCCUCGCAAtt |
| SEQ ID NO: 157 | AUCCAGGACAUCAUCAGUCtt |
| SEQ ID NO: 158 | UGCAAUCAAGUCGCUGUUAtt |
| SEQ ID NO: 159 | ACAUGCAAUCAAGUCGCUGtt |
| SEQ ID NO: 160 | CUGGACCAUGCCGCCAUCCtt |
| SEQ ID NO: 161 | CUGCAAUAGGCCUGCAAACtt |
| SEQ ID NO: 162 | CUGGAGGAUGGGGAAGGGCtt |
| SEQ ID NO: 163 | GCCUUUGGCCAGGCGCUGCtt |
| SEQ ID NO: 164 | CCUUGCCUGGAUGCGGCCUtt |
| SEQ ID NO: 165 | CUCUGCAUCUUCAUGCGCCtt |
| SEQ ID NO: 166 | GGCAGCUAUUCUCUGCAUCtt |
| SEQ ID NO: 167 | AGGUCCAGGAGGGCAGCUAtt |
| SEQ ID NO: 168 | ACUUCAGUGGUGGGCUGGAtt |
| SEQ ID NO: 169 | CGAGUCCAAACCCCAGGACtt |
| SEQ ID NO: 170 | CACGGCGAAGCCUGGCACCtt |
| SEQ ID NO: 171 | GACGGGGAGCUGUGCUGGU~~ |
| SEQ ID NO: 172 | GAAGGUGAGGCUUAGGCAGtt |
| SEQ ID NO: 173 | GCUUCCGUUUCGUUGACGAtt |
| SEQ ID NO: 174 | AGAGUCUGCUUCCGUUUCGtt |
| SEQ ID NO: 175 | GCUUAGAGUCUGCUUCCGUtt |
| SEQ ID NO: 176 | UGCUGGGCUUAGAGUCUGCtt |
| SEQ ID NO: 177 | GGCCACGUUCUUGCUGGGCtt |
| SEQ ID NO: 178 | GGGGGACAAGGGUCAGCGAtt |
| SEQ ID NO: 179 | GCUCAGCACAUGCAGCCUCtt |
| SEQ ID NO: 180 | AACGUGGGGCUUCUUCCCAtt |
| SEQ ID NO: 181 | UUCAAGGACAACGUGGGGCtt |
| SEQ ID NO: 182 | AUGCAAAGUGAAAAAGGAAtt |
| SEQ ID NO: 183 | GCCGGUGCAGUCAUCUGCAtt |
| SEQ ID NO: 184 | CAGUGACCUGGUCCAAUUCtt |
| SEQ ID NO: 185 | AGUCUGCUUUUCUACAAAUtt |
| SEQ ID NO: 186 | UGUUUAUCUAAGUCUGCUUtt |
| SEQ ID NO: 187 | GAUGUUUAUCUAAGUCUGCtt |
| SEQ ID NO: 188 | GAAGUUUUAAAAAUAAAUGtt |
| SEQ ID NO: 189 | CUUCUUUUAAAUAGAAGUUtt |
| SEQ ID NO: 190 | GACUUCUUUUAAAUAGAAGtt |
| SEQ ID NO: 191 | AUGACAUCAAACCUUAGUGtt |
| SEQ ID NO: 192 | UUUCACAUGACAUCAAACCtt |
| SEQ ID NO: 193 | AACUGUUAUUAUUACACUUtt |
| SEQ ID NO: 194 | UUAACUGUUAUUAUUACACtt |
| SEQ ID NO: 195 | GAAAUCUUAACUGUUAUUAtt |
| SEQ ID NO: 196 | CAUGAAAUCUUAACUGUUAtt |
| SEQ ID NO: 197 | GAUCAUGAAAUCUUAACUGtt |
| SEQ ID NO: 198 | UGAAAAUGAUCAUGAAAUC~~ |
| SEQ ID NO: 199 | GUGAGUAACAAAGAAUCACtt |
| SEQ ID NO: 200 | AUUCCACAGGCUUGCAGAGtt |
| SEQ ID NO: 201 | CCAGGAAGGUACUUCAUUAtt |
| SEQ ID NO: 202 | AAACUUUCCAGGAAGGUACtt |
| SEQ ID NO: 203 | UUAAAAAAUAAUCCAAACUtt |
| SEQ ID NO: 204 | ACAUGUAUCUCCCUUGUUUtt |
| SEQ ID NO: 205 | AUACAUGUAUCUCCCUUGUtt |
| SEQ ID NO: 206 | GAGAAUACAUGUAUCUCCC~~ |
| SEQ ID NO: 207 | CCUUUUCCUGGAGAAAUCUtt |
| SEQ ID NO: 208 | GCUGGCUACAUUCCUCCUU~~ |
| SEQ ID NO: 209 | GAGCUGGCUACAUUCCUCCtt |
| SEQ ID NO: 210 | GAGUGGGGAGCUGGCUACAtt |
| SEQ ID NO: 211 | AAGCUGUCUCUGUUUGGUUtt |
| SEQ ID NO: 212 | UGCUGGAAGCUGUCUCUGU~~ |
| SEQ ID NO: 213 | GGUCCCAACUGGUUCCUUCtt |
| SEQ ID NO: 214 | CACGGUCCCAACUGGUUCCtt |
| SEQ ID NO: 215 | GAGGGUGUCUUCCUUUGAUtt |
| SEQ ID NO: 216 | ACGUAGAGGGUGUCUUCCUtt |
| SEQ ID NO: 217 | AGGUGACGUAGAGGGUGUCtt |
| SEQ ID NO: 218 | CUCUGGGGACACCAUGCCCtt |
| SEQ ID NO: 219 | GAACCAUGGGGUUUCCAUAtt |
| SEQ ID NO: 220 | GAUGCAUCUGGGUCCUUGCtt |
| SEQ ID NO: 221 | CUGUGUUUCCUUCAUGAGGtt |
| SEQ ID NO: 222 | GCACAGCUACCCAGAGGGC~~ |
| SEQ ID NO: 223 | UCUAUGGAAGCAAAGGUCC~~ |
| SEQ ID NO: 224 | CCCCUUUCUGAGCUGUGCGtt |
| SEQ ID NO: 225 | UCUGCCCAUGUGGCCCCCUtt |
| SEQ ID NO: 226 | GUCGUGGUUUGUCCUUCCUtt |
| SEQ ID NO: 227 | CGGUGGUCGUGGUUUGUCC~~ |
| SEQ ID NO: 228 | UGGCCACGGUGGUCGUGGUtt |
| SEQ ID NO: 229 | CUUCCUUCUCUUCCAGGGAtt |
| SEQ ID NO: 230 | CCACCCUGCCUUCCUUCUCtt |
| SEQ ID NO: 231 | CCGCUCCACCCUGCCUUCCtt |
| SEQ ID NO: 232 | CCCCCCGCUCCACCCUGCCtt |
| SEQ ID NO: 233 | CCUUCUCUCCAUGAUGGUCtt |
| SEQ ID NO: 234 | UCUCCUGAUGCUGUGGUCCtt |
| SEQ ID NO: 235 | CCCUCUUGCUCUUAAAAAAtt |
| SEQ ID NO: 236 | CUCUCUACCCCUCUUGCUCtt |
| SEQ ID NO: 237 | UUGAUCCUCUCUACCCCUCtt |
| SEQ ID NO: 238 | AUCUCCAGCCAGGGCCAGC~~ |
| SEQ ID NO: 239 | GCCCCACAGACAGAGUCAUtt |
| SEQ ID NO: 240 | GCCACUUGCCUUCUCUAGUtt |
| SEQ ID NO: 241 | GGUGGGGCAGCCACUUGCCtt |
| SEQ ID NO: 242 | UGUUCCUCCUGGUCACGCCtt |
| SEQ ID NO: 243 | UGGAGUGAGCUGCAGGCUGtt |
| SEQ ID NO: 244 | CCAGUUGGAGCCCAGAGACtt |
| SEQ ID NO: 245 | UCAGUGGUUACAAGACCAGtt |
| SEQ ID NO: 246 | CUCCUUCAGUGCUCAGUGGtt |
| SEQ ID NO: 247 | CUGACCAAGACCUCUCUCCtt |
| SEQ ID NO: 248 | CUGGUCCUCCCAGUCACCUtt |
| SEQ ID NO: 249 | CUGCCCUGAGCAGUGUCUUtt |
| SEQ ID NO: 250 | CCCUGCCCUGAGCAGUGUCtt |
| SEQ ID NO: 251 | CAUGGGGACUGCCCUUUUCtt |
| SEQ ID NO: 252 | CCACAUGGGGACUGCCCUUtt |
| SEQ ID NO: 253 | GCCCACAUGGGGACUGCCC~~ |
| SEQ ID NO: 254 | GGUGUCCCCCAGACGCAAGtt |
| SEQ ID NO: 255 | CUCCUUACAUAAGCAAAGCtt |
| SEQ ID NO: 256 | GGCUGGCUCCCAGACCUCCtt |
| SEQ ID NO: 257 | AGCGCCCCAGCUAUGAGGUtt |
| SEQ ID NO: 258 | CUUCCCACACUCCUGGCUCtt |
| SEQ ID NO: 259 | ACAGCCCCCACUGUGGGCCtt |
| SEQ ID NO: 260 | AAGUGCUCUCUGCAGGGACtt |
| SEQ ID NO: 261 | GCCAGCCCUGCUCCCUGACtt |
| SEQ ID NO: 262 | CAAAGCCAAGGUUUGGGAGtt |
| SEQ ID NO: 263 | CAAUAUUCAAAGCCAAGGUtt |
| SEQ ID NO: 264 | GCACACCUCCACAACAAUA~~ |
| SEQ ID NO: 265 | ACCUGCUGGGACAGGUACCtt |
| SEQ ID NO: 266 | CGAUGGUGAAGGCUGGCCCtt |
| SEQ ID NO: 267 | GUGAACGCAAGUGUCUGGG~~ |
| SEQ ID NO: 268 | CACCUGCCCCUUAGGUUGCtt |
| SEQ ID NO: 269 | UCUUCACCUGCCCCUUAGGtt |
| SEQ ID NO: 270 | GCGUCUGGCAGGGCUGCGCtt |
| SEQ ID NO: 271 | ACGGUGCAUCUCAGAGACCtt |
| SEQ ID NO: 272 | AUCAGUUCACCCCACGCCUtt |
| SEQ ID NO: 273 | ACAAGAAGAUCAAAAUCAGtt |
| SEQ ID NO: 274 | AAUGCUUCAGAUUUAUUUAtt |
| SEQ ID NO: 275 | UUAAAUGCUUCAGAUUUAUtt |
| SEQ ID NO: 276 | UACAUUAAAUGCUUCAGAUtt |
| SEQ ID NO: 277 | AGAUGACUACAUUAAAUGCtt |
| SEQ ID NO: 278 | CAAUGUCAAGAUGACUACAtt |
| SEQ ID NO: 279 | AAAAAUUAUUUACAUAUUUtt |
| SEQ ID NO: 280 | UGCUGGCGAAAGCAGGUACtt |
| SEQ ID NO: 281 | UUUCAUUUCCACCCUCGUGtt |
| SEQ ID NO: 282 | AGGAAGUUCCAGUUUUCAUtt |
| SEQ ID NO: 283 | UUACAAGGAAGUUCCAGUUtt |
| SEQ ID NO: 284 | AUUUACAAGGAAGUUCCAGtt |
| SEQ ID NO: 285 | GUUUAAAUUUACAAGGAAGtt |
| SEQ ID NO: 286 | AUUUAAACUUGGCAAUAAAtt |
| SEQ ID NO: 287 | ACUUGGCAAUAAAAGAGAAtt |
| SEQ ID NO: 288 | CUUCUAUUUAAAAGAAGUCtt |
| SEQ ID NO: 289 | ACACCACAGUCACAGAACAtt |
| SEQ ID NO: 290 | UAUGUUUGUUUUCUUCUUGtt |
| SEQ ID NO: 291 | CAUCCUCUUUGAGAACAUCtt |
| SEQ ID NO: 292 | AUUCUAUCAUCGUUCAUAA~~ |
| SEQ ID NO: 293 | CACUUUCGUUAAAACUCAA~~ |
| SEQ ID NO: 294 | AACUCAACAACCACACUGUtt |
| SEQ ID NO: 295 | CUCAACAACCACACUGUCCtt |
| SEQ ID NO: 296 | AGCAUGGUUAGAAUUGCUGtt |
| SEQ ID NO: 297 | CCAUAACUUUGAAUGUCUAtt |
| SEQ ID NO: 298 | AACUGCUGUUCCUUACUUCtt |
| SEQ ID NO: 299 | CCAGGACAAGGGAGGAGAAtt |
| SEQ ID NO: 300 | UUAGCCUGCCGGUGUCUCU~~ |
| SEQ ID NO: 301 | GAGGCGGGUGCAAAAGAGAtt |
| SEQ ID NO: 302 | AAGAGACCCAACUACAAAAtt |
| SEQ ID NO: 303 | GAAGAAGAUGAGGAGAAAGtt |
| SEQ ID NO: 304 | GAAGAUGAGGAGAAAGGGCtt |
| SEQ ID NO: 305 | GGCUAAAGGUACAGAGGGUtt |
| SEQ ID NO: 306 | AGGUACAGAGGGUGGUUCAtt |
| SEQ ID NO: 307 | GAGAUCGAGAUGGUGAUCAtt |
| SEQ ID NO: 308 | UGGAGCAGACCCUUCCUGGtt |
| SEQ ID NO: 309 | AGACUCAUCAGGAUCAUGAtt |
| SEQ ID NO: 310 | CAACGCUGCCCAGCCAGAUtt |
| SEQ ID NO: 311 | UGAAGCCCAUGAACGUGGAtt |
| SEQ ID NO: 312 | CGUGGAAUAUCUCAUGAUGtt |
| SEQ ID NO: 313 | CAGGCACGCCACUGACGGGtt |
| SEQ ID NO: 314 | GCGGCUGCCGUGUGGCGAUtt |
| SEQ ID NO: 315 | GACCCGGCGGGCCAUCCGGtt |
| SEQ ID NO: 316 | UUGCGAGGGGAGCCUGAGAtt |
| SEQ ID NO: 317 | UCGUCAACGAAACGGAAGCtt |
| SEQ ID NO: 318 | CGAAACGGAAGCAGACUCUtt |
| SEQ ID NO: 319 | ACGGAAGCAGACUCUAAGCtt |
| SEQ ID NO: 320 | CGUGGCCAGGAGCGCAGCC~~ |
| SEQ ID NO: 321 | GAGGCUGCAUGUGCUGAGCtt |
| SEQ ID NO: 322 | GCCCCACGUUGUCCUUGAAtt |
| SEQ ID NO: 323 | GCAGACUUAGAUAAACAUCtt |
| SEQ ID NO: 324 | ACAUCUCCUUUGGAUAUUUtt |
| SEQ ID NO: 325 | CAUUUAUUUUUAAAACUUC~~ |
| SEQ ID NO: 326 | AACUUCUAUUUAAAAGAAGtt |
| SEQ ID NO: 327 | GUCCAAAAACAUCAACACUtt |
| SEQ ID NO: 328 | GGUUUGAUGUCAUGUGAAAtt |
| SEQ ID NO: 329 | AAGUGUAAUAAUAACAGUU~~ |
| SEQ ID NO: 330 | GUGUAAUAAUAACAGUUAAtt |
| SEQ ID NO: 331 | UAAUAACAGUUAAGAUUUCtt |
| SEQ ID NO: 332 | UAACAGUUAAGAUUUCAUGtt |
| SEQ ID NO: 333 | CAGUUAAGAUUUCAUGAUCtt |
| SEQ ID NO: 334 | GAUUUCAUGAUCAUUUUCAtt |
| SEQ ID NO: 335 | GUACCUUCCUGGAAAGUUUtt |
| SEQ ID NO: 336 | AGAUUUCUCCAGGAAAAGG~~ |
| SEQ ID NO: 337 | AAGGAGGAAUGUAGCCAGCtt |
| SEQ ID NO: 338 | GGAGGAAUGUAGCCAGCUCtt |
| SEQ ID NO: 339 | UGUAGCCAGCUCCCCACUCtt |
| SEQ ID NO: 340 | AACCAAACAGAGACAGCUUtt |
| SEQ ID NO: 341 | CCAAAC4GAGACAGCUUCCtt |
| SEQ ID NO: 342 | ACAGAGACAGCUUCCAGCAtt |
| SEQ ID NO: 343 | AUCAAAGGAAGACACCCUCtt |
| SEQ ID NO: 344 | CGCACAGCUCAGAAAGGGGtt |
| SEQ ID NO: 345 | AGGGGGCCACAUGGGCAGAtt |
| SEQ ID NO: 346 | ACCCAAAGGAAGGACAAACtt |
| SEQ ID NO: 347 | AGGAAGGACAAACCACGAC~~ |
| SEQ ID NO: 348 | GGACAAACCACGACCACCG~~ |
| SEQ ID NO: 349 | ACCACGACCACCGUGGCCAtt |
| SEQ ID NO: 350 | ACUAGAGAAGGCAAGUGGCtt |
| SEQ ID NO: 351 | GGCGUGACCAGGAGGAACAtt |
| SEQ ID NO: 352 | GCUUUGCUUAUGUAAGGAGtt |
| SEQ ID NO: 353 | GUCAGGGAGCAGGGCUGGCtt |
| SEQ ID NO: 354 | ACCUUGGCUUUGAAUAUUGtt |
| SEQ ID NO: 355 | GGGCCAGCCUUCACCAUCG~~ |
| SEQ ID NO: 356 | AUAAAUCUGAAGCAUUUAA~~ |
| SEQ ID NO: 357 | GGCGGCUCGCGGUUCCUCCtt |
| SEQ ID NO: 358 | GACUUCCCGCAACAUCCACtt |
| SEQ ID NO: 359 | CAUCCACUCCUUGGACCACtt |
| SEQ ID NO: 360 | GUAUCUGUACCACGUUUUGtt |
| SEQ ID NO: 361 | AAACACCACAGUCACAGAAtt |
| SEQ ID NO: 362 | CAGAACCGGAACCUGCUAGtt |
| SEQ ID NO: 363 | CCGGAACCUGCUAGUGGAGtt |
| SEQ ID NO: 364 | CCUGCUAGUGGAGACCAUCtt |
| SEQ ID NO: 365 | AAUGACAGCUCUGUAUUUGtt |
| SEQ ID NO: 366 | UGACAGCUCUGUAUUUGACtt |
| SEQ ID NO: 367 | GAAUAUGUUUGUUUUCUUCtt |
| SEQ ID NO: 368 | CAUCUUGCGGCAAAAGUCGtt |
| SEQ ID NO: 369 | AAGUCGGGCCGUUACGUGUtt |
| SEQ ID NO: 370 | GUCGGGCCGUUACGUGUGCtt |
| SEQ ID NO: 371 | CAUCAGUCACGAGACCUCAtt |
| SEQ ID NO: 372 | AUAACUACGUAAAUUCUAUtt |
| SEQ ID NO: 373 | CUACGUAAAUUCUAUCAUCtt |
| SEQ ID NO: 374 | AUUUGACUUUUCUGAUGAGtt |
| SEQ ID NO: 375 | AACACUUUCGUUAAAACUC~~ |
| SEQ ID NO: 376 | CAACCACACUGUCCAUUUCtt |
| SEQ ID NO: 377 | CCACACUGUCCAUUUCUUUtt |
| SEQ ID NO: 378 | UGAGCACACCAAUGACUUUtt |
| SEQ ID NO: 379 | UGACUUUGCCCUGUACACAtt |
| SEQ ID NO: 380 | GCCAUCAAGUUUUUCAACCtt |
| SEQ ID NO: 381 | GUUUUUCAACCACCCUGAAtt |
| SEQ ID NO: 382 | CCACCCUGAAAGCAUGGUUtt |
| SEQ ID NO: 383 | UUGCUGUAAGAACCAUAAC~~ |
| SEQ ID NO: 384 | GAACCAUAACUUUGAAUGUtt |
| SEQ ID NO: 385 | CUUUGAAUGUCUAUAAAGUtt |
| SEQ ID NO: 386 | UGUCUAUAAAGUGUCAUUGtt |
| SEQ ID NO: 387 | AGUGUCAUUGGAUAACCAGtt |
| SEQ ID NO: 388 | CCAGGCCAUGCUGCACUACtt |
| SEQ ID NO: 389 | CUGCUGUUCCUUACUUCUCtt |
| SEQ ID NO: 390 | UUUGGUCUGGUUCAUUGGGtt |
| SEQ ID NO: 391 | CUCGAUGACUGCGUGCAGAtt |
| SEQ ID NO: 392 | UCGGGGUAAACUGAGUGAUtt |
| SEQ ID NO: 393 | ACUGAGUGAUCUGGUGGCAtt |
| SEQ ID NO: 394 | UGACAUCCUGAUCAUCAACtt |
| SEQ ID NO: 395 | CUGUGAGUUCCUCAACGAUtt |
| SEQ ID NO: 396 | CGAUGUGCUCACUGACCACtt |
| SEQ ID NO: 397 | CAGGCUCUUCCUGCCCCUCtt |
| SEQ ID NO: 398 | GGGAGGAGAACGGCCGAAAtt |
| SEQ ID NO: 399 | CGGCCGAAAAUUAGCCUGCtt |
| SEQ ID NO: 400 | AAUUAGCCUGCCGGUGUCUtt |
| SEQ ID NO: 401 | UUAUACAUCAUGCACCGCUtt |
| SEQ ID NO: 402 | CUCGUUAGCUGAAGUCAUUtt |
| SEQ ID NO: 403 | GUCAUUCUGAAUGGUGAUCtt |
| SEQ ID NO: 404 | UGGUGAUCUGUCUGAGAUGtt |
| SEQ ID NO: 405 | GACUGAACAGGAUAUUCAGtt |
| SEQ ID NO: 406 | CAGGAUAUUCAGAGAAGUUtt |
| SEQ ID NO: 407 | GUUCUGCCAAGCCCAGCAUtt |
| SEQ ID NO: 408 | GCCCAGCAUUCGGUGCUUCtt |
| SEQ ID NO: 409 | ACCCACCGAGACACUCGAGtt |
| SEQ ID NO: 410 | CAAGCACAAGGGCAAGAGGtt |
| SEQ ID NO: 411 | GCACAAGGGCAAGAGGCGGtt |
| SEQ ID NO: 412 | GGGCAAGAGGCGGGUGCAAtt |
| SEQ ID NO: 413 | GAGACCCAACUACAAAAACtt |
| SEQ ID NO: 414 | CUACAAAAACGUUGGGGAAtt |
| SEQ ID NO: 415 | AAACGUUGGGGAAGAAGAAtt |
| SEQ ID NO: 416 | ACGUUGGGGAAGAAGAAGAtt |
| SEQ ID NO: 417 | GAUGAGGAGAAAGGGCCCAtt |
| SEQ ID NO: 418 | AGGGCCCACCGAGGAUGCCtt |
| SEQ ID NO: 419 | GAAGACGCCGAGAAGGCUAtt |
| SEQ ID NO: 420 | GACGCCGAGAAGGCUAAAGtt |
| SEQ ID NO: 421 | AAGGCAUCAAGACGAGUGGtt |
| SEQ ID NO: 422 | GGCAUCAAGACGAGUGGGGtt |
| SEQ ID NO: 423 | GACGAGUGGGGAGAGUGAAtt |
| SEQ ID NO: 424 | GCUCUCAGAGCUGGCCGCCtt |
| SEQ ID NO: 425 | CACCACGGACGAGGAGAAAtt |
| SEQ ID NO: 426 | AAGCGCCGCCGCCACCUGCtt |
| SEQ ID NO: 427 | GCGCCGCCGCCACCUGCUCtt |
| SEQ ID NO: 428 | UAAAGGCAUGGAUCCUGAAtt |
| SEQ ID NO: 429 | AGGCAUGGAUCCUGAAAAAtt |
| SEQ ID NO: 430 | AAAUUAGAGCGAAUCCAGCtt |
| SEQ ID NO: 431 | AUUAGAGCGAAUCCAGCUCtt |
| SEQ ID NO: 432 | UCCAGCUCCCCGUGCCAAAtt |
| SEQ ID NO: 433 | AUGCGGCCGAGAAGACCACtt |
| SEQ ID NO: 434 | GACCACCUACAACCACCCGtt |
| SEQ ID NO: 435 | CCACCCGCUAGCUGAAAGAtt |
| SEQ ID NO: 436 | CGCUGCCCAGCCAGAUGGGtt |
| SEQ ID NO: 437 | GAUCCGGCUGGCGACGCUGtt |
| SEQ ID NO: 438 | GCAGCAAGUCCUGAUGAGUtt |
| SEQ ID NO: 439 | GUCCUGAUGAGUGCUGGCUtt |
| SEQ ID NO: 440 | GGACGUGCACCUGGCCUGCtt |
| SEQ ID NO: 441 | GAAAGUGUUCACCUUGUACtt |
| SEQ ID NO: 442 | AGUGUUCACCUUGUACGACtt |
| SEQ ID NO: 443 | GGGAGAAGACAUUUUUUUGtt |
| SEQ ID NO: 444 | GACAUUUUUUUGGACAUGUtt |
| SEQ ID NO: 445 | GAUGAGUAUAGGAGCAUGAtt |
| SEQ ID NO: 446 | GCCCAUGAACGUGGAAUAUtt |
| SEQ ID NO: 447 | UAUCUCAUGAUGGACGCCUtt |
| SEQ ID NO: 448 | GACUGAUGAUGUCCUGGAUtt |
| SEQ ID NO: 449 | UAACAGCGACUUGAUUGCAtt |
| SEQ ID NO: 450 | CAGCGACUUGAUUGCAUGUtt |
| SEQ ID NO: 451 | GGAUGGCGGCAUGGUCCAGtt |
| SEQ ID NO: 452 | GUUUGCAGGCCUAUUGCAGtt |
| SEQ ID NO: 453 | CAUCACCAUCCACAAGCCUtt |
| SEQ ID NO: 454 | GCCUGCGUCCAGCCCCCAUtt |
| SEQ ID NO: 455 | GCCCUUCCCCAUCCUCCAGtt |
| SEQ ID NO: 456 | GCAGCGCCUGGCCAAAGGCtt |
| SEQ ID NO: 457 | AGGCCGCAUCCAGGCAAGGtt |
| SEQ ID NO: 458 | GGCGCAUGAAGAUGCAGAGtt |
| SEQ ID NO: 459 | GAUGCAGAGAAUAGCUGCCtt |
| SEQ ID NO: 460 | UAGCUGCCCUCCUGGACCUtt |
| SEQ ID NO: 461 | UCCAGCCCACCACUGAAGUtt |
| SEQ ID NO: 462 | GUCCUGGGGUUUGGACUCGtt |
| SEQ ID NO: 463 | GGUGCCAGGCUUCGCCGUGtt |
| SEQ ID NO: 464 | ACCAGCACAGCUCCCCGUCtt |
| SEQ ID NO: 465 | CUGCCUAAGCCUCACCUUCtt |
| SEQ ID NO: 466 | GCCUCACCUUCCUGACCAGtt |
| SEQ ID NO: 467 | GCAGACUCUAAGCCCAGCAtt |
| SEQ ID NO: 468 | GCCCAGCAAGAACGUGGCCtt |
| SEQ ID NO: 469 | GAACGUGGCCAGGAGCGCAtt |
| SEQ ID NO: 470 | UCGCUGACCCUUGUCCCCCtt |
| SEQ ID NO: 471 | GACACACUGGGAGCACCCAtt |
| SEQ ID NO: 472 | CCACCUAUCCCUGCGCUCCtt |
| SEQ ID NO: 473 | UGGGAAGAAGCCCCACGUUtt |
| SEQ ID NO: 474 | GAAGCCCCACGUUGUCCUUtt |
| SEQ ID NO: 475 | UUCCUUUUUCACUUUGCAUtt |
| SEQ ID NO: 476 | UGCAGAUGACUGCACCGGCtt |
| SEQ ID NO: 477 | GAAUUGGACCAGGUCACUGtt |
| SEQ ID NO: 478 | UUGGACCAGGUCACUGUACtt |
| SEQ ID NO: 479 | AUUUGUAGAAAAGCAGACUtt |
| SEQ ID NO: 480 | AAGCAGACUUAGAUAAACAtt |
| SEQ ID NO: 481 | AAGAAGUCCAAAAACAUCAtt |
| SEQ ID NO: 482 | GAAGUCCAAAAACAUCAACtt |
| SEQ ID NO: 483 | AAACAUCAACACUAAGGUUtt |
| SEQ ID NO: 484 | ACAUCAACACUAAGGUUUGtt |
| SEQ ID NO: 485 | CACUAAGGUUUGAUGUCAUtt |
| SEQ ID NO: 486 | GUGAUUCUUUGUUACUCACtt |
| SEQ ID NO: 487 | CUCUGCAAGCCUGUGGAAUtt |
| SEQ ID NO: 488 | GCCUGUGGAAUAAUGAAGUtt |
| SEQ ID NO: 489 | UAAUGAAGUACCUUCCUGGtt |
| SEQ ID NO: 490 | UGAAGUACCUUCCUGGAAAtt |
| SEQ ID NO: 491 | AGUUUGGAUUAUUUUUUAAtt |
| SEQ ID NO: 492 | ACAAAAACAAGGGAGAUACtt |
| SEQ ID NO: 493 | AAACAAGGGAGAUACAUGUtt |
| SEQ ID NO: 494 | ACAAGGGAGAUACAUGUAUtt |
| SEQ ID NO: 495 | GGGAGAUACAUGUAUUCUCtt |
| SEQ ID NO: 496 | UGGUUUUCUGCUAUAGCAGtt |
| SEQ ID NO: 497 | GAAGGAACCAGUUGGGACC~~ |
| SEQ ID NO: 498 | GGAACCAGUUGGGACCGUGtt |
| SEQ ID NO: 499 | CCAGUUGGGACCGUGAAGAtt |
| SEQ ID NO: 500 | GACUCCCGACCCUGUGGCCtt |
| SEQ ID NO: 501 | AGGAAGACACCCUCUACGUtt |
| SEQ ID NO: 502 | GACACCCUCUACGUCACCUtt |
| SEQ ID NO: 503 | GGGCAUGGUGUCCCCAGAGtt |
| SEQ ID NO: 504 | UAUGGAAACCCCAUGGUUCtt |
| SEQ ID NO: 505 | ACCCCAUGGUUCCCUUCCCtt |
| SEQ ID NO: 506 | GCAAGGACCCAGAUGCAUCtt |
| SEQ ID NO: 507 | GGACCCAGAUGCAUCAGACtt |
| SEQ ID NO: 508 | GAUGUUCCUUUCUACUCGGtt |
| SEQ ID NO: 509 | GUCCACCAGGGCCAGCGGCtt |
| SEQ ID NO: 510 | CUUUCCUCCAGACCUGCCAtt |
| SEQ ID NO: 511 | GAACAUGGUCUCUGUCUCCtt |
| SEQ ID NO: 512 | CAUGGUCUCUGUCUCCUCGtt |
| SEQ ID NO: 513 | GGCCUGCCGAGGGCAGUUUtt |
| SEQ ID NO: 514 | CCUCAUGAAGGAAACACAGtt |
| SEQ ID NO: 515 | GGAAACACAGUCCUGCCAAtt |
| SEQ ID NO: 516 | ACACAGUCCUGCCAAGGAGtt |
| SEQ ID NO: 517 | GGAGGGGGAGUGGCGCCCAtt |
| SEQ ID NO: 518 | GCCCUCUGGGUAGCUGUGCtt |
| SEQ ID NO: 519 | GGACCUUUGCUUCCAUAGAtt |
| SEQ ID NO: 520 | AACGCACAGCUCAGAAAGGtt |
| SEQ ID NO: 521 | UCCCUGGAAGAGAAGGAAGtt |
| SEQ ID NO: 522 | GAGAAGGAAGGCAGGGUGGtt |
| SEQ ID NO: 523 | GGAAGGCAGGGUGGAGCGGtt |
| SEQ ID NO: 524 | GGCAGGGUGGAGCGGGGGGtt |
| SEQ ID NO: 525 | GACCAUCAUGGAGAGAAGGtt |
| SEQ ID NO: 526 | GGACCACAGCAUCAGGAGAtt |
| SEQ ID NO: 527 | UUUUUUAAGAGCAAGAGGGtt |
| SEQ ID NO: 528 | GAGCAAGAGGGGUAGAGAGtt |
| SEQ ID NO: 529 | GAGGGGUAGAGAGGAUCAAtt |
| SEQ ID NO: 530 | GCUGGCCCUGGCUGGAGAUtt |
| SEQ ID NO: 531 | AUGACUCUGUCUGUGGGGCtt |
| SEQ ID NO: 532 | GGCAAGUGGCUGCCCCACCtt |
| SEQ ID NO: 533 | GUGGCUGCCCCACCCCAAGtt |
| SEQ ID NO: 534 | CAGCCUGCAGCUCACUCCAtt |
| SEQ ID NO: 535 | GUCUCUGGGCUCCAACUGGtt |
| SEQ ID NO: 536 | CUGGUCUUGUAACCACUGAtt |
| SEQ ID NO: 537 | CCACUGAGCACUGAAGGAGtt |
| SEQ ID NO: 538 | GGAGAGAGGUCUUGGUCAGtt |
| SEQ ID NO: 539 | AGGUGACUGGGAGGACCAGtt |
| SEQ ID NO: 540 | AAGACACUGCUCAGGGCAGtt |
| SEQ ID NO: 541 | GACACUGCUCAGGGCAGGGtt |
| SEQ ID NO: 542 | GAAAAGGGCAGUCCCCAUGtt |
| SEQ ID NO: 543 | AAGGGCAGUCCCCAUGUGGtt |
| SEQ ID NO: 544 | GGGCAGUCCCCAUGUGGGCtt |
| SEQ ID NO: 545 | CUUGCGUCUGGGGGACACCtt |
| SEQ ID NO: 546 | CCAGGGCUCCCAGAAGCUUtt |
| SEQ ID NO: 547 | GGAGGUCUGGGAGCCAGCCtt |
| SEQ ID NO: 548 | AGGACCCCACCUCACCCAGtt |
| SEQ ID NO: 549 | ACCUCAUAGCUGGGGCGCUtt |
| SEQ ID NO: 550 | GAGCCAGGAGUGUGGGAAGtt |
| SEQ ID NO: 551 | GGCCCACAGUGGGGGCUGUtt |
| SEQ ID NO: 552 | GUCCCUGCAGAGAGCACUUtt |
| SEQ ID NO: 553 | CUCCCAAACCUUGGCUUUGtt |
| SEQ ID NO: 554 | UAUUGUUGUGGAGGUGUGCtt |
| SEQ ID NO: 555 | GGUACCUGUCCCAGCAGGUtt |
| SEQ ID NO: 556 | CCCAGACACUUGCGUUCACtt |
| SEQ ID NO: 557 | GCAACCUAAGGGGCAGGUGtt |
| SEQ ID NO: 558 | CCUAAGGGGCAGGUGAAGA~~ |
| SEQ ID NO: 559 | GGGGCAGGUGAAGAAGCGCtt |
| SEQ ID NO: 560 | GAAGCGCAGCCCUGCCAGAtt |
| SEQ ID NO: 561 | GCGCAGCCCUGCCAGACGCtt |
| SEQ ID NO: 562 | GGUCUCUGAGAUGCACCGUtt |
| SEQ ID NO: 563 | AAAGGCGUGGGGUGAACUGtt |
| SEQ ID NO: 564 | AGGCGUGGGGUGAACUGAUtt |
| SEQ ID NO: 565 | CUGAUUUUGAUCUUCUUGUtt |
| SEQ ID NO: 566 | UAAAUAAAUCUGAAGCAUUtt |
| SEQ ID NO: 567 | AUCUGAAGCAUUUAAUGUAtt |
| SEQ ID NO: 568 | GCAUUUAAUGUAGUCAUCUtt |
| SEQ ID NO: 569 | UGUAGUCAUCUUGACAUUGtt |
| SEQ ID NO: 570 | AAAUAUGUAAAUAAUUUUUtt |
| SEQ ID NO: 571 | AUAUGUAAAUAAUUUUUGUtt |
| SEQ ID NO: 572 | AUAAUUUUUGUCCCAGUGAtt |
| SEQ ID NO: 573 | UUUUUGUCCCAGUGAGAACtt |
| SEQ ID NO: 574 | CCGAGGGUUAGAAAACCUCtt |
| SEQ ID NO: 575 | AACCUCGAUGCCUCUGAGCtt |
| SEQ ID NO: 576 | CCUCGAUGCCUCUGAGCCUtt |
| SEQ ID NO: 577 | GUACCUGCUUUCGCCAGCAtt |
| SEQ ID NO: 578 | CACGAGGGUGGAAAUGAAAtt |
| SEQ ID NO: 579 | AUGAAAACUGGAACUUCCUtt |
| SEQ ID NO: 580 | AACUGGAACUUCCUUGUAAtt |
| SEQ ID NO: 581 | CUGGAACUUCCUUGUAAAUtt |
| SEQ ID NO: 582 | CUUCCUUGUAAAUUUAAACtt |

Agents which reduce CLEC16A expression are not limited to siRNAs. Any agent that partially reduces CLEC16A expression is within the scope of the present invention.

### REFERENCES

Fenner, J. E., Starr, R., Cornish, A. L., Zhang, J. G., Metcalf, D., Schreiber, R. D., Sheehan, K., Hilton, D. J., Alexander, W. S., and Hertzog, P. J. (2006). Suppressor of cytokine signaling 1 regulates the immune response to infection by a unique inhibition of type I interferon activity. Nat Immunol 7, 33-39.
Khaibullin, T., Ivanova, V., Martynova, E., Cherepnev, G., Khabirov, F., Granatov, E., Rizvanov, A., and Khaiboullina, S. (2017). Elevated Levels of Proinflammatory Cytokines in Cerebrospinal Fluid of Multiple Sclerosis Patients. Front Immunol 8, 531.
Leikfoss, I. S., Mero, I. L., Dahle, M. K., Lie, B. A., Harbo, H. F., Spurkland, A., and Berge, T. (2013). Multiple sclerosis-associated single-nucleotide polymorphisms in CLEC16A correlate with reduced SOCS1 and DEXI expression in the thymus. Genes Immun 14, 62-66.
Redmann, V., Lamb, C. A., Hwang, S., Orchard, R. C., Kim, S., Razi, M., Milam, A., Park, S., Yokoyama, C. C., Kambal, A., et al. (2016). Clec16a is Critical for Autolysosome Function and Purkinje Cell Survival. Sci Rep 6, 23326.
Soleimanpour, S. A., Gupta, A., Bakay, M., Ferrari, A. M., Groff, D. N., Fadista, J., Spruce, L. A., Kushner, J. A., Groop, L., Seeholzer, S. H., et al. (2014). The diabetes susceptibility gene Clec16a regulates mitophagy. Cell 157, 1577-1590.
Tam, R. C., Li, M. W., Gao, Y. P., Pang, Y. T., Yan, S., Ge, W., Lau, C. S., and Chan, V. S. (2017). Human CLEC16A regulates autophagy through modulating mTOR activity. Exp Cell Res 352, 304-312.
Huang M, Verbeek D S. (2018). Why do so many genetic insults lead to Purkinje Cell degeneration and spinocerebellar ataxia? Neurosci Lett. 2018 February 5. pii: S0304-3940(18)30080-6

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A method of management for obesity in a subject in need thereof comprising administering to said subject a therapeutic agent in amount effective to partially reduce CLEC16A expression, thereby managing or reducing obesity.

2. The method of claim 1, wherein said therapeutic agent modulates signaling mediated via the CLEC16A gene product.

3. The method according to claim 1, wherein said therapeutic agent is selected from the group consisting of one or more of a small molecule, an antibody, a protein, an oligonucleotide, or an siRNA molecule.

4. The method of claim 1, wherein said therapeutic agent is an autophagy inhibitor.

5. The method of claim 1, wherein said agent is an inhibitor of the Jak-Stat pathway.

6. The method of claim 1, wherein said agent is an mTor inhibitor.

7. The method according to claim 3, wherein at least one siRNA molecule is set forth in Table 1.

8. The method of claim 1, wherein said therapeutic agent is delivered to an adipose cell.

9. The method of claim 1, wherein said therapeutic agent modulates natural killer cell activity.

10. The method of claim 1, wherein said therapeutic agent modulates signaling in an insulin-producing beta cell.

11. An siRNA composition comprising at least one nucleotide sequence selected from the group listed in Table 1 in a pharmaceutically acceptable carrier for delivery to a patient.

12. A method of partially inhibiting the expression of CLEC16A in a patient comprising administering to said patient at least one siRNA molecule that directs cleavage of a target CLEC16A mRNA sequence present in said patient.

13. The method of claim 12, wherein said siRNA is introduced directly into said patient.

14. The method of claim 7, wherein the siRNA is combined with other agents for treating obesity.

15. The method of claim 4, further comprising administration of a Jak-Stat inhibitor.

16. The method of claim 4, further comprising administration of a mTor inhibitor.

17. The method of claim 12, further comprising administration of a Jak-Stat inhibitor.

18. The method of claim 12, further comprising administration of a mTor inhibitor.

19. The method of claim 12, further comprising administration of an autophagy inhibitor.
